(19) United States
(12) Patent Application Publication
(10) Pub. No.: US 2022/0233691 A1
(43) Pub. Date: Jul. 28, 2022

(54) CELL LOCALIZATION SIGNATURE AND COMBINATION THERAPY

(71) Applicant: Bristol-Myers Squibb Company, Princeton, NJ (US)

(72) Inventors: Peter SZABO, Pennington, NJ (US); Lan ZHANG, Princeton, NJ (US); Keyur H. DESAI, Princeton, NJ (US); Neeraj ADYA, Princeton, NJ (US); Zhenhao QI, Princeton, NJ (US); Alex GREENFIELD, Princeton, NJ (US); George C. LEE, Princeton, NJ (US); Scott Adams ELY, Princeton, NJ (US); Saumya PANT, Princeton, NJ (US); George A. GREEN, Newton, NJ (US)

(73) Assignee: Bristol-Myers Squibb Company, Princeton, NJ (US)

(21) Appl. No.: 17/615,536

(22) PCT Filed: May 29, 2020
(86) PCT No.: PCT/US2020/035319

§ 371 Date: Nov. 30, 2021

Related U.S. Application Data
(60) Provisional application No. 62/854,887, filed on May 30, 2019.
(60) Provisional application No. 62/931,725, filed on Nov. 6, 2019.

(51) Int. Cl. A61K 39/395; A61P 35/04; A61K 45/06; G01N 33/574; C12Q 1/6886

(52) U.S. Cl. CPC A61K 39/3955; A61P 35/04; A61K 45/06; C12Q 1/6869; C12Q 1/6886; C12Q 2600/106; C12Q 2600/158; G01N 33/57492

(57) ABSTRACT

The present disclosure provides methods of identifying a subject suitable for an immuno-oncology (I-O) therapy comprising measuring the expression of one or more of STAT1, IFNγ, NECTIN2, and CSF1R. In some aspects, the I-O therapy comprises administering an anti-PD-1 antibody or antigen-binding portion thereof or an anti-PD-L1 antibody or antigen-binding portion thereof to the subject.

### CROSS REFERENCE TO RELATED APPLICATIONS

This PCT application claims the priority benefit of U.S. Provisional Application Nos. 62/854,887, filed May 30, 2019, and 62/931,725, filed Nov. 6, 2019, each of which is incorporated herein by reference in its entirety.

### FIELD OF THE DISCLOSURE

The present disclosure provides a method for treating a subject afflicted with a tumor using an immunotherapy.

### BACKGROUND OF THE DISCLOSURE

Human cancers harbor numerous genetic and epigenetic alterations, generating neoantigens potentially recognizable by the immune system (Sjoblom et al., Science (2006) 314(5797):268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

In the past decade, intensive efforts to develop specific immune checkpoint pathway inhibitors have begun to provide new immunotherapeutic approaches for treating cancer, including the development of antibodies that block the inhibitory Programmed Death-1 (PD-1)/Programmed Death ligand 1 (PD-L1) pathway such as nivolumab and pembrolizumab (formerly lambrolizumab; USAN Council Statement, 2013) that bind specifically to the PD-1 receptor and atezolizumab, durvalumab, and avelumab that bind specifically to PD-L1 (Topalian et al., 2012a, b; Topalian et al., 2014; Hamid et al., 2013; Hamid and Carvajal, 2013; McDermott and Atkins, 2013).

The immune system and response to immuno-therapy have shown to be complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

Certain aspects of the present disclosure are directed to a pharmaceutical composition comprising an anti-PD-1/PD-L1 antagonist for use in a method of identifying a human subject suitable for a combination therapy of the anti-PD-1/PD-L1 antagonist in combination with an anti-cancer agent, wherein the method comprises measuring expression of a panel of genes in a tumor sample obtained from a subject in need of the combination therapy, wherein the gene panel comprises at least three of CSF1R, NECTIN2, STAT1, and IFNγ. In some aspects, the gene panel comprises at least four, at least five, or at least six of CSF1R, NECTIN2, STAT1, and IFNγ. In some aspects, the gene panel comprises CSF1R, NECTIN2, STAT1, and IFNγ.

In some aspects, the subject is identified as being suitable when the tumor sample exhibits: (i) an increased expression of one or more of CSF1R and NECTIN2 (“upregulated genes”) in the sample compared to the expression of the one or more of CSF1R and NECTIN2 in a reference sample; (ii) a decreased expression of one or more of STAT1 and IFNγ (“down-regulated genes”) in the sample compared to the expression of one or more of STAT1 and IFNγ in a reference sample or (iii) both (i) and (ii). In some aspects, the subject is to be administered an anti-PD-1/PD-L1 antagonist in combination with an anti-cancer agent.

Certain aspects of the present disclosure are directed to a pharmaceutical composition comprising an anti-PD-1/PD-L1 antagonist in combination with an anti-cancer agent for use in a method of treating a human subject afflicted with a tumor, wherein a tumor sample obtained from the subject exhibits: (i) an increased expression of one or more of CSF1R and NECTIN2 (“upregulated genes”) in a tumor sample obtained from the subject compared to the expression of the one or more of CSF1R and NECTIN2 in a reference sample; (ii) a decreased expression of one or more of STAT1 and IFNγ (“down-regulated genes”) in a tumor sample obtained from the subject compared to the expression of one or more of STAT1 and IFNγ in a reference sample; or (iii) both (i) and (ii). In some aspects, the reference sample comprises a non-tumor tissue of the subject, a corresponding non-tumor tissue of the subject, or the corresponding tissue of subjects without a tumor.

Certain aspects of the present disclosure are directed to a method of identifying a human subject suitable for a combination therapy of an anti-PD-1/PD-L1 antagonist in combination with an anti-cancer agent, comprising in vitro measuring expression of a panel of genes in a tumor sample obtained from a subject in need of the anti-PD-1/PD-L1 antagonist, wherein the gene panel comprises at least three of CSF1R, NECTIN2, STAT1, and IFNγ. In some aspects, the gene panel comprises at least four, at least five, or at least six of CSF1R, NECTIN2, STAT1, and IFNγ. In some aspects, the gene panel comprises CSF1R, NECTIN2, STAT1, and IFNγ.

In some aspects, the subject is identified as being suitable when the tumor sample exhibits: (i) an increased expression of one or more of CSF1R and NECTIN2 (“upregulated genes”) in the tumor sample compared to the expression of the one or more of CSF1R and NECTIN2 in a reference sample; (ii) a decreased expression of one or more of STAT1 and IFNγ (“down-regulated genes”) in the tumor sample compared to the expression of one or more of STAT1 and IFNγ in a reference sample; or (iii) both (i) and (ii).

In some aspects, the method further comprises administering the anti-PD-1/PD-L1 antagonist in combination with an anti-cancer agent.

Certain aspects of the present disclosure are directed to a method of treating a human subject afflicted with a tumor, comprising administering an anti-PD-1/PD-L1 antagonist to the subject, wherein a tumor sample obtained from the subject exhibit: (i) an increased expression of one or more of CSF1R and NECTIN2 (“upregulated genes”) in a tumor sample obtained from the subject compared to the expression of the one or more of CSF1R and NECTIN2 in a reference sample; (ii) a decreased expression of one or more of STAT1 and IFNγ (“down-regulated genes”) in a tumor sample obtained from the subject compared to the expression of one or more of STAT1 and IFNγ in a reference sample; or (iii) both (i) and (ii). In some aspects, the reference sample comprises a non-tumor tissue of the subject, a corresponding non-tumor tissue of the subject, or the corresponding tissue of subjects without a tumor. In some aspects, the subject is identified as being suitable for an the anti-PD-1/PD-L1 antagonist prior to the anti-PD-1/PD-L1 antagonist. In some aspects, the tumor sample exhibits increased expression of at least two of the upregulated genes. In some aspects, the tumor sample exhibits decreased expression of at least two of the down-regulated genes. In some aspects, the tumor sample exhibits increased expression of all of the upregulated genes; and the tumor sample exhibits decreased expression of all of the down-regulated genes.

In some aspects, the expression of one or more of the upregulated genes is increased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than the expression of one or more of CSF1R and NECTIN2 in the reference sample. In some aspects, the expression of one or more of the upregulated genes is increased at least about 50% higher than the expression of one or more of CSF1R and NECTIN2 in the reference sample. In some aspects, the expression of one or more of the upregulated genes is increased at least about 75% higher than the expression of one or more of CSF1R and NECTIN2 in the reference sample.

In some aspects, the expression of one or more of the upregulated genes is decreased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% lower than the expression of one or more of STAT1 and IFNγ in the reference sample. In some aspects, the expression of one or more of the upregulated genes is decreased at least about 50% lower than the expression of one or more of STAT1 and IFNγ in the reference sample. In some aspects, the expression of one or more of the upregulated genes is decreased at least about 75% lower than the expression of one or more of STAT1 and IFNγ in the reference sample.

In some aspects, the tumor sample is a tumor tissue biopsy. In some aspects, the tumor sample is a formalin-fixed, paraffin-embedded tumor tissue or a fresh-frozen tumor tissue. In some aspects, the tumor sample is obtained from a stroma of the tumor.

In some aspects, gene expression is determined by detecting the presence of gene mRNA, the presence of a protein encoded by the gene, or both. In some aspects, the presence of gene mRNA is determined using reverse transcriptase PCR. In some aspects, the presence of the protein encoded by the gene is determined using an IHC assay. In some aspects, the IHC assay is an automated IHC assay. In some aspects, the tumor sample is obtained from a stroma of the tumor.

In some aspects, the anti-PD-1/PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody). In some aspects, the anti-PD-1/PD-L1 antagonist is an anti-PD-1 antibody. In some aspects, the anti-PD-1 antibody comprises nivolumab or pembrolizumab.

In some aspects, the anti-PD-1/PD-L1 antagonist is an anti-PD-L1 antibody. In some aspects, the anti-PD-1 antibody comprises avelumab, atezolizumab, or durvalumab.

In some aspects, the anti-cancer agent comprises an antibody that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CSF1R, CEACAM-1, CD52, HER2, and any combination thereof. In some aspects, the anti-cancer agent comprises an anti-CSF1R antibody.

In some aspects, the tumor is derived from a cancer selected from the group consisting of hepatocellular cancer, gastroesophageal cancer, melanoma, bladder cancer, lung cancer, kidney cancer, head and neck cancer, colon cancer, pancreatic cancer, prostate cancer, ovarian cancer, urothelial cancer, colorectal cancer, and any combination thereof.

In some aspects, the tumor is relapsed. In some aspects, the tumor is refractory. In some aspects, the tumor is locally advanced. In some aspects, the tumor is metastatic. In some aspects, the administering treats the tumor.

In some aspects, the administering reduces the size of the tumor. In some aspects, the size of the tumor is reduced by at least about 10%, about 20%, about 30%, about 40%, or about 50% compared to the tumor size prior to the administration.

In some aspects, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some aspects, the subject exhibits stable disease after the administration. In some aspects, the subject exhibits a partial response after the administration. In some aspects, the subject exhibits a complete response after the administration.

Certain aspects of the present disclosure are directed to a kit for treating a subject afflicted with a tumor, the kit comprising: (a) an anti-PD-1/PD-L1 antagonist; and (b) instructions for using the anti-PD-1/PD-L1 antagonist in the pharmaceutical composition in combination with an anti-cancer agent disclosed herein or a method disclosed herein. In some aspects, the anti-PD-1/PD-L1 antagonist comprises an anti-PD-1 antibody. In some aspects, the anti-PD-1/PD-L1 antagonist comprises an anti-PD-L1 antibody. In some aspects, the anti-cancer agent comprises an antibody that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CSF1R, CEACAM-1, CD52, HER2, and any combination thereof.

Certain aspects of the present disclosure are directed to a gene panel comprising at least three of CSF1R, NECTIN2, STAT1, and IFNγ, for use in identifying a subject suitable for a combination therapy comprising an anti-PD-1/PD-L1 antagonist and an anti-cancer agent. In some aspects, the gene panel comprises at least four, at least five, or at least six of CSF1R, NECTIN2, STAT1, and IFNγ. In some aspects, the gene panel comprises CSF1R, NECTIN2, STAT1, and IFNγ. In some aspects, the gene panel consists of CSF1R, NECTIN2, STAT1, and IFNγ and one additional gene, two additional genes, three additional genes, four additional genes, five additional genes, six additional genes, seven additional genes, eight additional genes, nine additional genes, or ten additional genes.

A method for preparing a nucleic acid fraction from a tumor of a subject in need of an I/O therapy, comprising: (a) extracting a tumor biopsy from the subject; (b) producing a fraction of nucleic acids extracted in (a) by isolating the nucleic acids; and (c) analyzing the expression level of one or more genes in a gene panel selected from STAT1, IFNγ, NECTIN2, and CSF1R. In some aspects, the nucleic acids are mRNA.

In some aspects, one or both of CSF1R and NECTIN2 genes are upregulated. In some aspects, one or both of STAT1 and IFNγ genes are downregulated. In some aspects, CSF1R and NECTIN2 are upregulated, and STAT1 and IFNγ are downregulated.

In some aspects, the expression level of the one or more genes in the gene panel is analyzed by measuring an mRNA level of the one or more genes in the gene panel in the tumor sample. In some aspects, the expression level is measured using a nuclease protection assay. In some aspects, the expression level is measured using next-generation sequencing. In some aspects, the expression level is measured using reverse transcriptase polymerase chain reaction (RT-PCR).

In some aspects, the expression of one or both of STAT1 and IFNγ is decreased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% lower than the expression of one or both of STAT1 and IFNγ in the reference sample. In some aspects, the expression of one or both of STAT1 and IFNγ is decreased at least about 50% lower than the expression of one or both of STAT1 and IFNγ in the reference sample. In some aspects, the expression of one or both of STAT1 and IFNγ is decreased at least about 75% lower than the expression of one or both of STAT1 and IFNγ in the reference sample.

In some aspects, the expression of one or both of NECTIN2 and CSF1R is increased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than the expression of one or more of NECTIN2 and CSF1R in the reference sample. In some aspects, the expression of one or both of NECTIN2 and CSF1R is increased at least about 50% higher than the expression of one or more of NECTIN2 and CSF1R in the reference sample. In some aspects, the expression of one or both of NECTIN2 and CSF1R is increased at least about 75% higher than the expression of one or more of NECTIN2 and CSF1R in the reference sample.

### BRIEF DESCRIPTION OF THE DRAWINGS

FIGS. 1A-1B are images of tumor tissue samples labeled using standard CD8+ immunohistochemistry (IHC; FIG. 1A) or further annotated using an artificial intelligence (AI) image analysis tool (FIG. 1B). Arrowheads are examples of CD8+ T cells. Tumor parenchyma and stromal regions are indicated (FIG. 1B).

FIG. 2 is a schematic illustration of the various CD8 phenotypes.

FIGS. 3A-3B are scatter plots illustrating the cutoffs for parenchymal vs. stromal abundance used to define immune phenotypes. FIG. 3B includes the scatter plot of FIG. 3A with an overlay indicating the cutoffs for inflamed, balanced, excluded, and desert phenotypes.

FIGS. 4A-4C are images of three tumor samples, which are representative of an immune desert phenotype (T cells absent from the TME; FIG. 4A), an immune excluded phenotype (accumulated T cells without efficient tumor parenchyma infiltration; FIG. 4B), and an immune inflamed phenotype (infiltrated T cells in the tumor parenchyma; FIG. 4C). Arrowheads label examples of CD8+ T cells.

FIGS. 5A-5D are graphical representations, illustrating the parenchymal CD8 signature (FIGS. 5A-5B) and the stromal CD8 signature (FIGS. 5C-5D). FIGS. 5A and 5C are heat maps showing the relative expressions of various genes that are associated with a parenchymal CD8 signature (FIG. 5A) and the stromal CD8 signature (FIG. 5C). FIGS. 5B and 5D are bar graph summaries of select representative genes of the parenchymal CD8 signature (FIG. 5B) and the stromal CD8 signature (FIG. 5D).

FIGS. 6A-6D are scatter plots, illustrating the correlation of CD8 signature scores with CD8 IHC scores in melanoma (FIGS. 6A and 6C) and SCCHN (FIGS. 6B and 6D) tumor samples. FIGS. 6A-6B show the correlation between the parenchymal AI-based CD8 IHC score (y-axis) and the parenchymal CD8 signature score (x-axis) (adjusted R²=0.67). FIGS. 6C-6D show the correlation between the stromal AI-based CD8 IHC score (y-axis) and the stromal CD8 signature score (x-axis) (adjusted R²=0.65). The x=y and linear regression lines are overlaid onto each graph. Adjusted R² values are derived from pooled analyses.

FIG. 7A is graphical representation of the overall survival (OS) odds ratios for Triple CD8, Dual CD8, Parenchymal CD8, CD8, and CD8.IHC_EMT, as indicated. FIGS. 7B-7F are ROC curves for OR for Triple CD8 (FIG. 7B), Dual CD8 (FIG. 7D, Parenchymal CD8 (FIG. 7C), CD8 (FIG. 7E), and CD8.IHC_EMT (FIG. 7F).

FIG. 8A is graphical representation of the progression free survival (PFS) odds ratios for Triple CD8, Dual CD8, Parenchymal CD8, CD8, and CD8.IHC_EMT, as indicated. FIG. 8B is graphical representation of the OS odds ratios for Triple CD8, Dual CD8, Parenchymal CD8, CD8, and CD8.IHC_EMT, as indicated.

FIGS. 9A-9B are graphical representations of PFS (FIG. 9A) and OS (FIG. 9B) as stratified by parenchymal signature score. The number of patients at risk in each group is shown below each graph.

### DETAILED DESCRIPTION OF THE DISCLOSURE

Certain aspects of the present disclosure are directed to methods of identifying a human subject suitable for an immune-oncology (I-O) therapy, e.g., an anti-PD-1/PD-L1 antagonist therapy. In some aspects, the present disclosure is directed to a method of identifying a human subject suitable for an anti-PD-1/PD-L1 antagonist therapy, comprising measuring expression of a panel of genes in a tumor sample obtained from a subject in need of the anti-PD-1/PD-L1 antagonist, wherein the gene panel comprises at least one of STAT1, IFNγ, NECTIN2, and CSF1R. A gene panel comprising the identified genes of the present disclosure and the gene signature are useful to identify a subject suitable for and/or responsive to an I-O therapy in combination with an anticancer agent, especially in predicting an inflammatory phenotype in the tumor microenvironment (TME) across multiple tumor types. Therefore, in some aspects, the gene panel and its use can replace the inconvenient and burdensome CD8+ immunohistochemistry.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

“Administering” refers to the physical introduction of a composition comprising a therapeutic agent to a subject, using any of the various methods and delivery systems known to those skilled in the art. Preferred routes of administration for the immunotherapy, e.g., the anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, the term “anti-PD-1 antibody” includes a full antibody having two heavy chains and two light chains that specifically binds to PD-1 and antigen-binding portions of the full antibody. Non limiting examples of the antigen-binding portions are shown elsewhere herein.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1, an anti-PD-L1 antibody binds specifically to PD-L1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody or an anti-PD-L1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

Antibodies useful in the methods and compositions described herein include, but are not limited to, antibodies and antigen-binding portions thereof that specifically bind a protein selected from the group consisting of Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), Cytotoxic T-Lymphocyte Antigen-4 (CTLA-4), B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, IL-2, B7-H4, Fas ligand, CXCR4, CSF1R, mesothelin, CEACAM-1, CD52, HER2, MICA, MICB, CSF1R, and any combination thereof.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

As used herein, a PD-1 or PD-L1 “inhibitor,” refers to any molecule capable of blocking, reducing, or otherwise limiting the interaction between PD-1 and PD-L1 and/or the activity of PD-1 and/or PD-L1. In some aspects, the inhibitor is an antibody or an antigen-binding fragment of an antibody. In other aspects, the inhibitor comprises a small molecule.

“Signal transducer and activator of transcription 1-alpha/beta” or “STAT1,” as used herein, refers to a signal transducer and transcription activator that mediates cellular responses to interferons (IFNs), cytokine KITLG/SCF, and other cytokines and other growth factors. Following type I IFN (IFN-alpha and IFN-beta) binding to cell surface receptors, signaling via protein kinases leads to activation of Jak kinases (TYK2 and JAK1) and to tyrosine phosphorylation of STAT1 and STAT2. The phosphorylated STATs dimerize and associate with ISGF3G/IRF-9 to form a complex termed ISGF3 transcription factor, that enters the nucleus. ISGF3 binds to the IFN stimulated response element (ISRE) to activate the transcription of IFN-stimulated genes (ISG), which drive the cell in an antiviral state. In response to type II IFN (IFN-gamma), STAT1 is tyrosine- and serine-phosphorylated. It then forms a homodimer termed IFN-gamma-activated factor (GAF), migrates into the nucleus and binds to the IFN gamma activated sequence (GAS) to drive the expression of the target genes, inducing a cellular antiviral state. STAT1 becomes activated in response to KITLG/SCF and KIT signaling. STAT1 may also mediate cellular responses to activated FGFR1, FGFR2, FGFR3, and FGFR4. The complete human STAT1 amino acid sequence can be found under UniProtKB identification number P42224. The human STAT1 protein is encoded by the human STAT1 gene (NCBI Gene ID: 6772).

“Interferon gamma,” “IFN-gamma,” or “IFNγ,” as used herein, refers to a cytokine that is involved in innate and adaptive immunity against infection (UniProtKB—P01579). IFNγ is the only member of the type II class of interferons, and is therefore sometimes referred to as “type II interferon.” IFNγ serves to activate macrophages and induce MHC class II expression. IFNγ is largely expressed by natural killer (NK) cells and natural killer T (NKT) cells as part of the innate immune response, and by CD4 Thl (T helper) cells and CD8 cytotoxic T lymphocyte (CTL) effector T cells once immunity develops.

“NECTIN2,” as used herein, refers to the gene encoding Nectin-2, which is a modulator of T cell signaling, serving as both a co-stimulator and a co-inhibitor of T cell function, depending on which receptor that Nectin-2 binds (UniProtKB—Q92692). Binding of Nectin-2 to CD226 stimulates T cell proliferation and production of various cytokines including IL2, IL5, IL10, IL13, and IFNγ. Conversely, binding of Nectin-2 to PVRIG inhibits T cell proliferation.

“CSF1R,” as used herein, refers to the gene encoding macrophage colony-stimulating factor 1 receptor (CSF-1-R; UniProtKB—P07333), a tyrosine-protein kinase with multiple functions. In particular, CSF-1-R acts as a cell-surface receptor for CSF1 and IL34 and plays an essential role in the regulation of survival, proliferation, and differentiation of hematopoietic precursor cells, especially mononuclear phagocytes, such as macrophages and monocytes. In addition, binding of IL34 or CSF1 to CSF-1-R promotes the release of proinflammatory chemokines involved in innate immunity and the inflammatory process.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, an “immuno-oncology” therapy or an “I-O” therapy refers to a therapy that comprises utilizing an immune response to target and treat a tumor in a subject. As such, as used herein, an I-O therapy is a type of anti-cancer therapy. In some aspects, and I-O therapy comprises administering an antibody or an antigen-binding fragment thereof to a subject. In some aspects, an I-O therapy comprises administering to a subject an immune cell, e.g., a T cell, e.g., a modified T cell, e.g., a T cell modified to express a chimeric antigen receptor or an particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony stimulating factor to a subject.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred aspects of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some aspects, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some aspects, a therapeutically effective amount or dosage of the drug completely inhibits cell growth or tumor growth, i.e., inhibits cell growth or tumor growth by 100%. The ability of a compound to inhibit tumor growth can be evaluated using an assay described herein. Alternatively, this property of a composition can be evaluated by examining the ability of the compound to inhibit cell growth, such inhibition can be measured in vitro by assays known to the skilled practitioner. In some aspects described herein, tumor regression can be observed and continue for a period of at least about 20 days, at least about 40 days, or at least about 60 days.

The term “biological sample” as used herein refers to biological material isolated from a subject. The biological sample can contain any biological material suitable for determining target gene expression, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one aspect, the sample is a tumor sample. In some aspects, the tumor sample can be obtained from a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

A “tumor sample,” as used herein, refers to a biological sample that comprises tumor tissue. In some aspects, a tumor sample is a tumor biopsy. In some aspects, a tumor sample comprises tumor cells and one or more non-tumor cell present in the tumor microenvironment (TME). For the purposes of the present disclosure, the TME is made up of at least two regions. The tumor “parenchyma” is a region of the TME that includes predominantly tumor cells, e.g., the part (or parts) of the TME that includes the bulk of the tumor cells. The tumor parenchyma does not necessarily consist of only tumor cells, rather other cells such as stromal cells and/or lymphocytes can also be present in the parenchyma. The “stromal” region of the TME includes the adjacent non-tumor cells. In some aspects, the tumor sample comprises all or part of the tumor parenchyma and one or more cells of the stroma. In some aspects, the tumor sample is obtained from the parenchyma. In some aspects the tumor sample is obtained from the stroma. In other aspects, the tumor sample is obtained from the parenchyma and the stroma.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of can mean within 1 or more than 1 standard deviation per the practice in the art. Alternatively, “about” or “comprising essentially of can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Disclosure

Inflammation in the TME can be an indicator of potential responsiveness to an I-O therapy in combination with an anticancer agent. However, contemporary methods for measuring inflammation in a tumor require the laborious process of immunohistochemistry to detect and analyze CD8 expression in a tumor biopsy. It was surprisingly found that the expression pattern of a relatively small number of genes (in some aspects, at least about 4 genes) correlates with inflammation in a tumor microenvironment. In some aspects, the methods described herein can replace the need for time-consuming IHC. Some aspects of the present disclosure are directed to methods of identifying a human subject suitable for an I-O therapy, e.g., an anti-PD-1/PD-L1 antagonist, comprising measuring expression of a panel of genes in a tumor sample obtained from a subject in need of the anti-PD-1/PD-L1 antagonist, wherein the gene panel comprises at least one of STAT1, IFNγ, NECTIN2, and CSF1R. In some aspects the measuring is conducted in vitro.

Some aspects of the present disclosure are directed to a method for preparing a nucleic acid fraction from a tumor of a subject in need of an I/O therapy, comprising: (a) extracting a tumor biopsy from the subject; (b) producing a fraction of nucleic acids extracted in (a) by isolating the nucleic acids; and (c) analyzing the expression level of one or more genes in a gene panel selected from STAT1, IFNγ, NECTIN2, and CSF1R. In some aspects, the nucleic acids are mRNA.

In certain aspects, the gene panel comprises at least two of STAT1, IFNγ, NECTIN2, and CSF1R. In some aspects, the gene panel comprises STAT1 and IFNγ. In some aspects, the gene panel comprises STAT1 and NECTIN2. In some aspects, the gene panel comprises STAT1 and CSF1R. In some aspects, the gene panel comprises IFNγ and NECTIN2. In some aspects, the gene panel comprises IFNγ and CSF1R. In some aspects, the gene panel comprises NECTIN2 and CSF1R.

In certain aspects, the gene panel comprises at least three of STAT1, IFNγ, NECTIN2, and CSF1R. In some aspects, the gene panel comprises STAT1, IFNγ, and NECTIN2. In some aspects, the gene panel comprises STAT1, IFNγ, and CSF1R. In some aspects, the gene panel comprises STAT1, NECTIN2, and CSF1R. In some aspects, the gene panel comprises IFNγ, NECTIN2, and CSF1R.

In certain aspects, the gene panel comprises STAT1, IFNγ, NECTIN2, and CSF1R. In some aspects, the gene panel further comprises one or more additional genes. In some aspects, the gene panel comprises at least 2 to at least about 100 genes. In some aspects, the gene panel comprises at least 2 to at least about 95, at least 2 to at least about 90, at least 2 to at least about 85, at least 2 to at least about 80, at least 2 to at least about 75, at least 2 to at least about 70, at least 2 to at least about 65, at least 2 to at least about 60, at least 2 to at least about 55, at least 2 to at least about 50, at least 2 to at least about 45, at least 2 to at least about 40, at least 2 to at least about 35, at least 2 to at least about 30, at least 2 to at least about 25, at least 2 to at least about 20, at least 2 to at least about 15, at least 2 to at least about 10, at least 2 to at least about 9, at least 2 to at least about 8, at least 2 to at least about 7, at least 2 to at least about 6, at least 2 to at least about 5, or at least 2 to at least about 4 genes.

In some aspects, the gene panel comprises at least 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 genes.

In some aspects the gene panel consists of STAT1, IFNγ, NECTIN2, and CSF1R. In some aspects, the gene panel consists essentially of STAT1, IFNγ, NECTIN2, and CSF1R. In some aspects, the gene panel consists or consists essentially of STAT1, IFNγ, NECTIN2, and CSF1R and at least 1 additional gene, at least 2 additional genes, at least 3 additional genes, at least 4 additional genes, at least 5 additional genes, at least 6 additional genes, at least 7 additional genes, at least 8 additional genes, at least 9 additional genes, at least 10 additional genes, at least 11 additional genes, at least 12 additional genes, at least 13 additional genes, at least 14 additional genes, at least 15 additional genes, at least 20 additional genes, at least 25 additional genes, at least 30 additional genes, at least 35 additional genes, at least 40 additional genes, at least 45 additional genes, at least 50 additional genes, at least 55 additional genes, at least 60 additional genes, at least 65 additional genes, at least 70 additional genes, at least 75 additional genes, at least 80 additional genes, at least 85 additional genes, at least 90 additional genes, at least 95 additional genes, or at least 100 additional genes.

II.A. Gene Expression Profiling

Gene expression profiling, as used herein, is a measurement of the combined expression level the genes in a panel disclosed herein, e.g., comprising, consisting essentially of, or consisting of at least one, at least two, or at least three of STAT1, IFNγ, NECTIN2, and CSF1R. In some aspects, the measurement is made using a sample obtained from a subject. In certain aspects, the sample is a tumor sample. Any biological sample comprising one or more tumor cell can be used in the methods disclosed herein. In some aspects, the sample is selected from a tumor biopsy, a blood sample, a serum sample, or any combination thereof. In certain aspects, the sample is a tumor biopsy collected from the subject prior to administration of a therapy described herein, e.g., an I-O therapy, e.g., an anti-PD-1/PD-L1 agonist. In particular aspects, the sample obtained from the subject is a formalin-fixed tumor biopsy. In some aspects, the sample obtained from the subject is a paraffin-embedded tumor biopsy. In some aspects, the sample obtained from the subject is a fresh-frozen tumor biopsy.

Any method known in the art for measuring the expression of a particular gene or a panel of genes can be used in the methods of the present disclosure. In some aspects, the expression of one or more of the inflammatory genes in the inflammatory gene panel is determined by detecting the presence of mRNA transcribed from the inflammatory gene, the presence of a protein encoded by the inflammatory gene, or both.

In some aspects, the expression a gene is determined by measuring the level of gene mRNA, e.g., by measuring the level of one or more of STAT1 mRNA, IFNγ mRNA, NECTIN2 mRNA, and CSF1R mRNA, in a sample obtained from the subject. In certain aspects, the gene expression profile is determined by measuring the level of STAT1 mRNA, IFNγ mRNA, NECTIN2 mRNA, CSF1R mRNA, or any combination thereof in a sample obtained from the subject. Any method known in the art can be used to measure the level of the gene mRNA. In some aspects, the gene mRNA is measured using reverse transcriptase PCR. In some aspects, the gene mRNA is measured using a nuclease protection assay. In some aspects, the gene mRNA is measured using next-generation sequencing (NGS). In some aspects, the gene mRNA is measured using RNA in situ hybridization.

In some aspects, the expression of a gene is determined by measuring the level of protein encoded by the gene, e.g., by measuring the level of one or more of STAT1 protein, IFNγ protein, NECTIN2 protein, and CSF1R protein, in a sample obtained from the subject. In certain aspects, the gene expression profile is determined by measuring the level of STAT1 protein, IFNγ protein, NECTIN2 protein, CSF1R protein, or any combination thereof in a sample obtained from the subject. Any method known in the art can be used to measure the level of the protein. In some aspects, the gene expression profile is measured using an immunohistochemistry (IHC) assay. In certain aspects, the IHC is an automated IHC.

In some aspects, the expression of one or more of the genes of the gene panel is normalized relative to the expression of one or more housekeeping genes. In some aspects, the one or more housekeeping genes are made up of genes that have relatively consistent expression across various tumor types in various subjects.

In some aspects, raw gene expression values are normalized following standard gene expression profiling protocols. In these aspects, a gene expression profile can be calculated as the median or average of the log 2-transformed normalized and scaled expression values across all of the target genes in the signature, and presented on a linear scale. In certain aspects, profiles have positive or negative values, depending on whether gene expression is up- or down-regulated under a particular condition.

In certain aspects, increased/decreased expression is characterized by an expression level that is greater/less than the expression of the same gene or genes in a reference sample. In some aspects, the reference sample comprises a non-tumor tissue of the same subject. In some aspects, the reference sample comprises a corresponding non-tumor tissue of the same subject. In some aspects the reference sample comprises a corresponding tissue in a subject that does not have a tumor. In some aspects, the reference sample comprises more than one tumor tissue sample from more than one other subject, e.g., the increased expression is relative to an average expression level across more than one other tumor samples.

In some aspects, the increased/decreased expression is characterized by an expression level that is greater/less than a reference expression level. In some aspects, the reference expression level is an average expression level. In some aspects, the average expression level is determined by measuring the expression of the gene (or the multiple genes) present in the gene panel in tumor samples obtained from a population of subjects, and calculating the average for the population of subjects. In some aspects, each member of the population of subjects is afflicted with the same tumor as the subject being administered the I-O therapy, e.g., an anti-PD-1/PD-L1 antagonist.

In some aspects, increased expression of the upregulated genes, e.g., STAT1 or IFNγ for parenchymal gene signature or NECTIN2 and CSF1R for stromal gene signature, is characterized by an expression level that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than an expression level in a reference sample or than an average expression level. In certain aspects, increased expression is characterized by an expression level that is at least about 25% higher than an expression level in a reference sample or than an average expression level. In certain aspects, increased expression is characterized by an expression level that is at least about 30% higher than an expression level in a reference sample or than an average expression level. In certain aspects, increased expression is characterized by an expression level that is at least about 35% higher than an expression level in a reference sample or than an average expression level. In certain aspects, increased expression is characterized by an expression level that is at least about 40% higher than an expression level in a reference sample or than an average expression level. In certain aspects, increased expression is characterized by an expression level that is at least about 45% higher than an expression level in a reference sample or than an average expression level. In certain aspects, increased expression is characterized by an expression level that is at least about 50% higher than an expression level in a reference sample or than an average expression level. In certain aspects, increased expression is characterized by an expression level that is at least about 55% higher than an expression level in a reference sample or than an average expression level. In certain aspects, increased expression is characterized by an expression level that is at least about 60% higher than an expression level in a reference sample or than an average expression level. In certain aspects, increased expression is characterized by an expression level that is at least about 65% higher than an expression level in a reference sample or than an average expression level. In certain aspects, increased expression is characterized by an expression level that is at least about 70% higher than an expression level in a reference sample or than an average expression level. In certain aspects, increased expression is characterized by an expression level that is at least about 75% higher than an expression level in a reference sample or than an average expression level. In certain aspects, increased expression is characterized by an expression level that is at least about 80% higher than an expression level in a reference sample or than an average expression level. In certain aspects, increased expression is characterized by an expression level that is at least about 85% higher than an expression level in a reference sample or than an average expression level. In certain aspects, increased expression is characterized by an expression level that is at least about 90% higher than an expression level in a reference sample or than an average expression level. In certain aspects, increased expression is characterized by an expression level that is at least about 95% higher than an expression level in a reference sample or than an average expression level. In certain aspects, increased expression is characterized by an expression level that is at least about 100% higher than an expression level in a reference sample or than an average expression level. In certain aspects, increased expression is characterized by an expression level that is at least about 125% higher than an expression level in a reference sample or than an average expression level. In certain aspects, increased expression is characterized by an expression level that is at least about 150% higher than an expression level in a reference sample or than an average expression level. In certain aspects, increased expression is characterized by an expression level that is at least about 175% higher than an expression level in a reference sample or than an average expression level. In certain aspects, increased expression is characterized by an expression level that is at least about 200% higher than an expression level in a reference sample or than an average expression level. In certain aspects, increased expression is characterized by an expression level that is at least about 225% higher than an expression level in a reference sample or than an average expression level. In certain aspects, increased expression is characterized by an expression level that is at least about 250% higher than an expression level in a reference sample or than an average expression level. In certain aspects, increased expression is characterized by an expression level that is at least about 275% higher than an expression level in a reference sample or than an average expression level. In certain aspects, increased expression is characterized by an expression level that is at least about 300% higher than an expression level in a reference sample or than an average expression level.

In some aspects, increased expression of the upregulated genes, e.g., STAT1 or IFNγ for parenchymal gene signature or NECTIN2 and CSF1R for stromal gene signature, is characterized by an expression level that is at least about 1.25-fold, at least about 1.30-fold, at least about 1.35-fold, at least about 1.40-fold, at least about 1.45-fold, at least about 1.50-fold, at least about 1.55-fold, at least about 1.60-fold, at least about 1.65-fold, at least about 1.70-fold, at least about 1.75-fold, at least about 1.80-fold, at least about 1.85-fold, at least about 1.90-fold, at least about 1.95-fold, at least about 2-fold, at least about 2.25-fold, at least about 2.50-fold, at least about 2.75-fold, at least about 3-fold, at least about 3.25-fold, at least about 3.50-fold, at least about 3.75-fold, or at least about 400-fold higher than an expression level in a reference sample or than an average expression level. In certain aspects, increased expression is characterized by an expression level that is at least about 1.25-fold higher than an expression level in a reference sample or than an average expression level. In certain aspects, increased expression is characterized by an expression level that is at least about 1.30-fold higher than an expression level in a reference sample or than an average expression level. In certain aspects, increased expression is characterized by an expression level that is at least about 1.35-fold higher than an expression level in a reference sample or than an average expression level. In certain aspects, increased expression is characterized by an expression level that is at least about 1.40-fold higher than an expression level in a reference sample or than an average expression level. In certain aspects, increased expression is characterized by an expression level that is at least about 1.45-fold higher than an expression level in a reference sample or than an average expression level. In certain aspects, increased expression is characterized by an expression level that is at least about 1.50-fold higher than an expression level in a reference sample or than an average expression level. In certain aspects, increased expression is characterized by an expression level that is at least about 1.55-fold higher than an expression level in a reference sample or than an average expression level. In certain aspects, increased expression is characterized by an expression level that is at least about 1.60-fold higher than an expression level in a reference sample or than an average expression level. In certain aspects, increased expression is characterized by an expression level that is at least about 1.65-fold higher than an expression level in a reference sample or than an average expression level. In certain aspects, increased expression is characterized by an expression level that is at least about 1.70-fold higher than an expression level in a reference sample or than an average expression level. In certain aspects, increased expression is characterized by an expression level that is at least about 1.75-fold higher than an expression level in a reference sample or than an average expression level. In certain aspects, increased expression is characterized by an expression level that is at least about 1.80-fold higher than an expression level in a reference sample or than an average expression level. In certain aspects, increased expression is characterized by an expression level that is at least about 1.85-fold higher than an expression level in a reference sample or than an average expression level. In certain aspects, increased expression is characterized by an expression level that is at least about 1.90-fold higher than an expression level in a reference sample or than an average expression level. In certain aspects, increased expression is characterized by an expression level that is at least about 1.95-fold higher than an expression level in a reference sample or than an average expression level. In certain aspects, increased expression is characterized by an expression level that is at least about 2-fold higher than an expression level in a reference sample or than an average expression level. In certain aspects, increased expression is characterized by an expression level that is at least about 2.25-fold higher than an expression level in a reference sample or than an average expression level. In certain aspects, increased expression is characterized by an expression level that is at least about 2.50-fold higher than an expression level in a reference sample or than an average expression level. In certain aspects, increased expression is characterized by an expression level that is at least about 2.75-fold higher than an expression level in a reference sample or than an average expression level. In certain aspects, increased expression is characterized by an expression level that is at least about 3-fold higher than an expression level in a reference sample or than an average expression level. In certain aspects, increased expression is characterized by an expression level that is at least about 3.25-fold higher than an expression level in a reference sample or than an average expression level. In certain aspects, increased expression is characterized by an expression level that is at least about 3.50-fold higher than an expression level in a reference sample or than an average expression level. In certain aspects, increased expression is characterized by an expression level that is at least about 3.75-fold higher than an expression level in a reference sample or than an average expression level. In certain aspects, increased expression is characterized by an expression level that is at least about 4-fold higher than an expression level in a reference sample or than an average expression level.

In certain aspects, decreased expression of the down-regulated genes, e.g., NECTIN2 and CSF1R for parenchymal gene signature or STAT1 or IFNγ for stromal gene signature, is characterized by an expression level that is less than a reference expression level. In some aspects, decreased expression is characterized by an expression level that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% lower than an expression level in a reference sample or than an average expression level. In certain aspects, decreased expression is characterized by an expression level that is at least about 25% lower than an expression level in a reference sample or than an average expression level. In certain aspects, decreased expression is characterized by an expression level that is at least about 30% lower than an expression level in a reference sample or than an average expression level. In certain aspects, decreased expression is characterized by an expression level that is at least about 35% lower than an expression level in a reference sample or than an average expression level. In certain aspects, decreased expression is characterized by an expression level that is at least about 40% lower than an expression level in a reference sample or than an average expression level. In certain aspects, decreased expression is characterized by an expression level that is at least about 45% lower than an expression level in a reference sample or than an average expression level. In certain aspects, decreased expression is characterized by an expression level that is at least about 50% lower than an expression level in a reference sample or than an average expression level. In certain aspects, decreased expression is characterized by an expression level that is at least about 55% lower than an expression level in a reference sample or than an average expression level. In certain aspects, decreased expression is characterized by an expression level that is at least about 60% lower than an expression level in a reference sample or than an average expression level. In certain aspects, decreased expression is characterized by an expression level that is at least about 65% lower than an expression level in a reference sample or than an average expression level. In certain aspects, decreased expression is characterized by an expression level that is at least about 70% lower than an expression level in a reference sample or than an average expression level. In certain aspects, decreased expression is characterized by an expression level that is at least about 75% lower than an expression level in a reference sample or than an average expression level. In certain aspects, decreased expression is characterized by an expression level that is at least about 80% lower than an expression level in a reference sample or than an average expression level. In certain aspects, decreased expression is characterized by an expression level that is at least about 85% lower than an expression level in a reference sample or than an average expression level. In certain aspects, decreased expression is characterized by an expression level that is at least about 90% lower than an expression level in a reference sample or than an average expression level. In certain aspects, decreased expression is characterized by an expression level that is at least about 95% lower than an expression level in a reference sample or than an average expression level. In certain aspects, decreased expression is characterized by an expression level that is at least about 100% lower than an expression level in a reference sample or than an average expression level. In certain aspects, decreased expression is characterized by an expression level that is at least about 125% lower than an expression level in a reference sample or than an average expression level. In certain aspects, decreased expression is characterized by an expression level that is at least about 150% lower than an expression level in a reference sample or than an average expression level. In certain aspects, decreased expression is characterized by an expression level that is at least about 175% lower than an expression level in a reference sample or than an average expression level. In certain aspects, decreased expression is characterized by an expression level that is at least about 200% lower than an expression level in a reference sample or than an average expression level. In certain aspects, decreased expression is characterized by an expression level that is at least about 225% lower than an expression level in a reference sample or than an average expression level. In certain aspects, decreased expression is characterized by an expression level that is at least about 250% lower than an expression level in a reference sample or than an average expression level. In certain aspects, decreased expression is characterized by an expression level that is at least about 275% lower than an expression level in a reference sample or than an average expression level. In certain aspects, decreased expression is characterized by an expression level that is at least about 300% lower than an expression level in a reference sample or than an average expression level.

In some aspects, decreased expression of the down-regulated genes, e.g., NECTIN2 and CSF1R for parenchymal gene signature or STAT1 or IFNγ for stromal gene signature, is characterized by an expression level that is at least about 1.25-fold, at least about 1.30-fold, at least about 1.35-fold, at least about 1.40-fold, at least about 1.45-fold, at least about 1.50-fold, at least about 1.55-fold, at least about 1.60-fold, at least about 1.65-fold, at least about 1.70-fold, at least about 1.75-fold, at least about 1.80-fold, at least about 1.85-fold, at least about 1.90-fold, at least about 1.95-fold, at least about 2-fold, at least about 2.25-fold, at least about 2.50-fold, at least about 2.75-fold, at least about 3-fold, at least about 3.25-fold, at least about 3.50-fold, at least about 3.75-fold, or at least about 400-fold lower than an expression level in a reference sample or than an average expression level. In certain aspects, decreased expression is characterized by an expression level that is at least about 1.25-fold lower than an expression level in a reference sample or than an average expression level. In certain aspects, decreased expression is characterized by an expression level that is at least about 1.30-fold lower than an expression level in a reference sample or than an average expression level. In certain aspects, decreased expression is characterized by an expression level that is at least about 1.35-fold lower than an expression level in a reference sample or than an average expression level. In certain aspects, decreased expression is characterized by an expression level that is at least about 1.40-fold lower than an expression level in a reference sample or than an average expression level. In certain aspects, decreased expression is characterized by an expression level that is at least about 1.45-fold lower than an expression level in a reference sample or than an average expression level. In certain aspects, decreased expression is characterized by an expression level that is at least about 1.50-fold lower than an expression level in a reference sample or than an average expression level. In certain aspects, decreased expression is characterized by an expression level that is at least about 1.55-fold lower than an expression level in a reference sample or than an average expression level. In certain aspects, decreased expression is characterized by an expression level that is at least about 1.60-fold lower than an expression level in a reference sample or than an average expression level. In certain aspects, decreased expression is characterized by an expression level that is at least about 1.65-fold lower than an expression level in a reference sample or than an average expression level. In certain aspects, decreased expression is characterized by an expression level that is at least about 1.70-fold lower than an expression level in a reference sample or than an average expression level. In certain aspects, decreased expression is characterized by an expression level that is at least about 1.75-fold lower than an expression level in a reference sample or than an average expression level. In certain aspects, decreased expression is characterized by an expression level that is at least about 1.80-fold lower than an expression level in a reference sample or than an average expression level. In certain aspects, decreased expression is characterized by an expression level that is at least about 1.85-fold lower than an expression level in a reference sample or than an average expression level. In certain aspects, decreased expression is characterized by an expression level that is at least about 1.90-fold lower than an expression level in a reference sample or than an average expression level. In certain aspects, decreased expression is characterized by an expression level that is at least about 1.95-fold lower than an expression level in a reference sample or than an average expression level. In certain aspects, decreased expression is characterized by an expression level that is at least about 2-fold lower than an expression level in a reference sample or than an average expression level. In certain aspects, decreased expression is characterized by an expression level that is at least about 2.25-fold lower than an expression level in a reference sample or than an average expression level. In certain aspects, decreased expression is characterized by an expression level that is at least about 2.50-fold lower than an expression level in a reference sample or than an average expression level. In certain aspects, decreased expression is characterized by an expression level that is at least about 2.75-fold lower than an expression level in a reference sample or than an average expression level. In certain aspects, decreased expression is characterized by an expression level that is at least about 3-fold lower than an expression level in a reference sample or than an average expression level. In certain aspects, decreased expression is characterized by an expression level that is at least about 3.25-fold lower than an expression level in a reference sample or than an average expression level. In certain aspects, decreased expression is characterized by an expression level that is at least about 3.50-fold lower than an expression level in a reference sample or than an average expression level. In certain aspects, decreased expression is characterized by an expression level that is at least about 3.75-fold lower than an expression level in a reference sample or than an average expression level. In certain aspects, decreased expression is characterized by an expression level that is at least about 4-fold lower than an expression level in a reference sample or than an average expression level.

II.B. Methods of Treatment

Certain aspects of the present disclosure are directed to methods of identifying a subject suitable for a therapy and then administering the therapy to the suitable subject. The methods of identifying a suitable subject described herein can be used in advance of any immuno-oncology (I-O) therapy. In some aspects, the suitable subject is to be administered and/or subsequently administered an antibody or antigen-binding fragment thereof that specifically binds a protein selected from PD-1, PD-L1, CTLA-4, LAG-3, TIGIT, TIM3, CSF1R, NKG2a, OX40, ICOS, CD137, KIR, TGFβ, IL-10, IL-8, IL-2, CD96, VISTA, B7-H4, Fas ligand, CXCR4, mesothelin, CD27, GITR, MICA, MICB, and any combination thereof.

In some aspects, the suitable subject is to be administered and/or subsequently administered an antibody or antigen-binding fragment thereof that specifically binds PD-1. In some aspects, the suitable subject is to be administered and/or subsequently administered an antibody or antigen-binding fragment thereof that specifically binds PD-L1. In some aspects, the suitable subject is to be administered and/or subsequently administered an antibody or antigen-binding fragment thereof that specifically binds CTLA-4. In some aspects, the suitable subject is to be administered and/or subsequently administered an antibody or antigen-binding fragment thereof that specifically binds LAG-3. In some aspects, the suitable subject is to be administered and/or subsequently administered an antibody or antigen-binding fragment thereof that specifically binds TIGIT. In some aspects, the suitable subject is to be administered and/or subsequently administered an antibody or antigen-binding fragment thereof that specifically binds TIM3. In some aspects, the suitable subject is to be administered and/or subsequently administered an antibody or antigen-binding fragment thereof that specifically binds GITR. In some aspects, the suitable subject is to be administered and/or subsequently administered an antibody or antigen-binding fragment thereof that specifically binds MICA. In some aspects, the suitable subject is to be administered and/or subsequently administered an antibody or antigen-binding fragment thereof that specifically binds MICB. In some aspects, the suitable subject is to be administered and/or subsequently administered an antibody or antigen-binding fragment thereof that specifically binds CSF1R.

In some aspects, the suitable subject is to be administered and/or subsequently administered more than one antibody or antigen-binding fragment thereof disclosed herein. In some aspects, the suitable subject is to be administered and/or subsequently administered at least two antibodies or antigen-binding fragments thereof. In some aspects, the suitable subject is to be administered and/or subsequently administered at least three antibodies or antigen-binding fragments thereof. In certain aspects the suitable subject is to be administered and/or subsequently administered an antibody or antigen-binding fragment thereof that specifically binds PD-1 and an antibody or antigen-binding fragment thereof that specifically binds CTLA-4. In certain aspects the suitable subject is to be administered and/or subsequently administered an antibody or antigen-binding fragment thereof that specifically binds PD-L1 and an antibody or antigen-binding fragment thereof that specifically binds CTLA-4. In certain aspects the suitable subject is to be administered and/or subsequently administered an antibody or antigen-binding fragment thereof that specifically binds PD-1 and an antibody or antigen-binding fragment thereof that specifically binds CSF1R. In certain aspects the suitable subject is to be administered and/or subsequently administered an antibody or antigen-binding fragment thereof that specifically binds PD-L1 and an antibody or antigen-binding fragment thereof that specifically binds CSF1R. In certain aspects the suitable subject is to be administered and/or subsequently administered an antibody or antigen-binding fragment thereof that specifically binds PD-1 and an antibody or antigen-binding fragment thereof that specifically binds LAG-3. In certain aspects the suitable subject is to be administered and/or subsequently administered an antibody or antigen-binding fragment thereof that specifically binds PD-L1 and an antibody or antigen-binding fragment thereof that specifically binds LAG-3.

In certain aspects, the therapy is administered to the suitable subject after the gene expression profile has been measured. In some aspects, the measuring is in vitro. In other aspects, the measuring is in vivo. In some aspects, the therapy is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days after the gene expression profile has been measured.

In some aspects, the particular therapy to be administered and/or subsequently administered to the suitable subject is dependent on the gene expression profile. In certain aspects, the gene expression profile has a parenchymal signature. In other aspects, the gene expression profile has a stromal signature.

II.B.1. Parenchymal Signature

In some aspects, the present disclosure is directed to a pharmaceutical composition comprising an I-O therapy, e.g., an anti-PD-1/PD-L1 antagonist, for use in a method of identifying a human subject suitable for the anti-PD-1/PD-L1 antagonist, wherein the method comprises measuring expression of a panel of genes in a tumor sample obtained from a subject in need of the anti-PD-1/PD-L1 antagonist, wherein the gene panel comprises at least three or four of STAT1, IFNγ, NECTIN2, and CSF1R. In some aspects, the subject is identified as being suitable when the tumor sample exhibits:
(i) an increased expression of one or more of STAT1 and IFNγ (“upregulated genes”) in the sample compared to the expression of the one or more of STAT1 and IFNγ in a reference sample;
(ii) a decreased expression of one or more of NECTIN2 and CSF1R (“down-regulated genes”) in the sample compared to the expression of one or more of NECTIN2 and CSF1R in a reference sample or
(iii) both (i) and (ii). In other aspects, the subject is to be administered an anti-PD-1/PD-L1 antagonist.

The present disclosure provides, in some aspects, a pharmaceutical composition comprising an I-O therapy, e.g., an anti-PD-1/PD-L1 antagonist, for use in a method of treating a human subject afflicted with a tumor, wherein a tumor sample obtained from the subject exhibits:
(i) an increased expression of one or more of STAT1 and IFNγ (“upregulated genes”) in a tumor sample obtained from the subject compared to the expression of the one or more of STAT1 and IFNγ in a reference sample;
(ii) a decreased expression of one or more of NECTIN2 and CSF1R (“down-regulated genes”) in a tumor sample obtained from the subject compared to the expression of one or more of NECTIN2 and CSF1R in a reference sample; or
(iii) both (i) and (ii).

In other aspects, the disclosure provides a method of identifying a human subject suitable for an anti-PD-1/PD-L1 antagonist therapy, comprising measuring expression of a panel of genes in a tumor sample obtained from a subject in need of the anti-PD-1/PD-L1 antagonist, wherein the gene panel comprises at least three of STAT1, IFNγ, NECTIN2, and CSF1R. In some aspects, the measuring is in vitro. In other aspects, the measuring is in vivo.

As used herein, the subject has a parenchymal signature when the subject exhibits (i) an increased expression of one or more of STAT1 and IFNγ (“upregulated genes”) in a tumor sample obtained from the subject compared to the expression of the one or more of STAT1 and IFNγ in a reference sample; (ii) a decreased expression of one or more of NECTIN2 and CSF1R (“down-regulated genes”) in a tumor sample obtained from the subject compared to the expression of one or more of NECTIN2 and CSF1R in a reference sample; or (iii) both (i) and (ii).

As such, a subject having a parenchymal signature can be identified as being suitable for an I-O therapy, e.g., an anti-PD-1/PD-L1 antagonist therapy. As used herein a subject having a parenchymal signature has a tumor characterized by (i) increased expression of STAT1 and/or IFNγ (“upregulated genes”), (ii) decreased expression of NECTIN2 and/or CSF1R (“down-regulated genes”), or (ii) both (i) and (ii). Thus, in some aspects, the subject is identified as being suitable when the tumor sample exhibits: (i) an increased expression of STAT1 and/or IFNγ (“upregulated genes”) in the tumor sample compared to the expression of STAT1 and/or IFNγ in a reference sample; (ii) a decreased expression of NECTIN2 and/or CSF1R (“down-regulated genes”) in the tumor sample compared to the expression of one or more of NECTIN2 and/or CSF1R in a reference sample; or (iii) both (i) and (ii). In some aspects, a suitable subject has a tumor characterized by increased expression of STAT1. In some aspects, a suitable subject has a tumor characterized by increased expression of IFNγ. In some aspects, a suitable subject has a tumor characterized by increased expression of STAT1 and IFNγ. In some aspects, a suitable subject has a tumor characterized by decreased expression of NECTIN2. In some aspects, a suitable subject has a tumor characterized by decreased expression of CSF1R. In some aspects, a suitable subject has a tumor characterized by decreased expression of NECTIN2 and CSF1R. In some aspects, a suitable subject has a tumor characterized by increased expression of STAT1 and decreased expression of NECTIN2. In some aspects, a suitable subject has a tumor characterized by increased expression of STAT1 and decreased expression of NECTIN2. In some aspects, a suitable subject has a tumor characterized by increased expression of IFNγ and decreased expression of NECTIN2. In some aspects, a suitable subject has a tumor characterized by increased expression of IFNγ and decreased expression of CSF1R. In some aspects, a suitable subject has a tumor characterized by increased expression of STAT1 and IFNγ and decreased expression of NECTIN2 and CSF1R.

In certain aspects, the suitable subject, e.g., the subject having a parenchymal signature described herein, is to be administered and/or subsequently administered an I-O therapy described herein. In some aspects, the suitable subject, e.g., the subject having a parenchymal signature described herein, is to be administered and/or subsequently administered an anti-PD-1/PD-L1 antagonist. In some aspects, the suitable subject, e.g., the subject having a parenchymal signature described herein, is to be administered and/or subsequently administered an anti-PD-1 antibody. In some aspects, the suitable subject, e.g., the subject having a parenchymal signature described herein, is to be administered and/or subsequently administered an anti-PD-L1 antibody. In some aspects, the suitable subject, e.g., the subject having a parenchymal signature described herein, is to be administered and/or subsequently administered an anti-PD-1 antibody monotherapy. In some aspects, the suitable subject, e.g., the subject having a parenchymal signature described herein, is to be administered and/or subsequently administered an anti-PD-L1 antibody monotherapy. In some aspects, the suitable subject, e.g., the subject having a parenchymal signature described herein, is to be administered and/or subsequently administered a combination therapy comprising an anti-PD-1 antibody and one or more additional anti-cancer agents (e.g., one or more additional I-O therapy described herein, one or more chemotherapy, or any combination thereof).

In certain aspects, an anti-PD-1/PD-L1 antagonist is administered to a subject, wherein a tumor sample obtained from the subject exhibits: (i) an increased expression of STAT1 and IFNγ (“upregulated genes”) in a tumor sample obtained from the subject compared to the expression of STAT1 and IFNγ in a reference sample; (ii) a decreased expression of NECTIN2 and CSF1R (“down-regulated genes”) in a tumor sample obtained from the subject compared to the expression of one or more of NECTIN2 and CSF1R in a reference sample; or (iii) both (i) and (ii).

In some aspects, an anti-PD-1/PD-L1 antagonist is administered to a subject, wherein a tumor sample obtained from the subject does not exhibit: (i) a decreased expression of STAT1 or IFNγ (“upregulated genes”) in a tumor sample obtained from the subject compared to the expression of STAT1 or IFNγ in a reference sample; (ii) an increased expression of NECTIN2 or CSF1R (“down-regulated genes”) in a tumor sample obtained from the subject compared to the expression of one or more of NECTIN2 and CSF1R in a reference sample; or (iii) both (i) and (ii).

II.B.2. Stromal Signature

In some aspects, the tumor sample from a subject does not exhibit parenchymal gene signature, but rather exhibits a gene signature showing: (i) an increased expression of one or more of NECTIN2 and CSF1R (“upregulated genes”) in the sample compared to the expression of the one or more of NECTIN2 and CSF1R in a reference sample; (ii) a decreased expression of one or more of STAT1 and IFNγ (“down-regulated genes”) in the sample compared to the expression of one or more of STAT1 and IFNγ in a reference sample or (iii) both (i) and (ii), e.g., stromal signature. In some aspects, the subject with a stromal gene signature is not suitable for an I-O monotherapy, e.g. an anti-PD-1/PD-L1 antagonist monotherapy. In other aspects, the subject with a stromal gene signature is suitable for a combination therapy of an I-O therapy and an anti-cancer agent.

In some aspects, the disclosure provides a pharmaceutical composition comprising an anti-PD-1/PD-L1 antagonist for use in a method of identifying a human subject suitable for a combination therapy of the anti-PD-1/PD-L1 antagonist in combination with an anti-cancer agent, wherein the method comprises measuring expression of a panel of genes in a tumor sample obtained from a subject in need of the combination therapy, wherein the gene panel comprises at least three of CSF1R, NECTIN2, STAT1, and IFNγ.

In some aspects, the subject is identified as being suitable when the tumor sample exhibits: (i) an increased expression of one or more of CSF1R and NECTIN2 (“upregulated genes”) in the sample compared to the expression of the one or more of CSF1R and NECTIN2 in a reference sample; (ii) a decreased expression of one or more of STAT1 and IFNγ (“down-regulated genes”) in the sample compared to the expression of one or more of STAT1 and IFNγ in a reference sample or (iii) both (i) and (ii). In other aspects, the subject is to be administered an anti-PD-1/PD-L1 antagonist in combination with an anti-cancer agent.

In some aspects, the disclosure provides a pharmaceutical composition comprising an anti-PD-1/PD-L1 antagonist in combination with an anti-cancer agent for use in a method of treating a human subject afflicted with a tumor, wherein a tumor sample obtained from the subject exhibits: (i) an increased expression of one or more of CSF1R and NECTIN2 (“upregulated genes”) in a tumor sample obtained from the subject compared to the expression of the one or more of CSF1R and NECTIN2 in a reference sample; (ii) a decreased expression of one or more of STAT1 and IFNγ (“down-regulated genes”) in a tumor sample obtained from the subject compared to the expression of one or more of STAT1 and IFNγ in a reference sample; or (iii) both (i) and (ii).

In other aspects, the disclosure is directed to a method of identifying a human subject suitable for a combination therapy of an anti-PD-1/PD-L1 antagonist in combination with an anti-cancer agent, comprising measuring expression of a panel of genes in a tumor sample obtained from a subject in need of the anti-PD-1/PD-L1 antagonist, wherein the gene panel comprises at least three of CSF1R, NECTIN2, STAT1, and IFNγ. In other aspects, the measuring is in vivo. In some aspects, the measuring is in vitro. In some aspects, the subject is identified as being suitable when the tumor sample exhibits: (i) an increased expression of one or more of CSF1R and NECTIN2 (“upregulated genes”) in the tumor sample compared to the expression of the one or more of CSF1R and NECTIN2 in a reference sample; (ii) a decreased expression of one or more of STAT1 and IFNγ (“down-regulated genes”) in the tumor sample compared to the expression of one or more of STAT1 and IFNγ in a reference sample; or (iii) both (i) and (ii). In some aspects, the method further comprises administering the anti-PD-1/PD-L1 antagonist in combination with an anti-cancer agent.

As used herein, the subject has a stromal signature when the subject exhibits (i) an increased expression of one or more of CSF1R and NECTIN2 (“upregulated genes”) in the tumor sample compared to the expression of the one or more of CSF1R and NECTIN2 in a reference sample; (ii) a decreased expression of one or more of STAT1 and IFNγ (“down-regulated genes”) in the tumor sample compared to the expression of one or more of STAT1 and IFNγ in a reference sample; or (iii) both (i) and (ii). In some aspects, a subject having a stromal signature is identified as being suitable for a combination therapy comprising (i) an I-O therapy, e.g., anti-PD-1/PD-L1 antagonist therapy and (ii) an additional anti-cancer therapy. As used herein a subject having a stromal signature has a tumor characterized by (i) decreased expression of STAT1 and/or IFNγ (“down-regulated genes”), (ii) increased expression of NECTIN2 and/or CSF1R (“upregulated genes”), or (ii) both (i) and (ii). Thus, in some aspects, the subject is identified as being suitable when the tumor sample exhibits: (i) a decreased expression of STAT1 and/or IFNγ (“down-regulated genes”) in the tumor sample compared to the expression of STAT1 and/or IFNγ in a reference sample; (ii) an increased expression of NECTIN2 and/or CSF1R (“upregulated genes”) in the tumor sample compared to the expression of NECTIN2 and/or CSF1R in a reference sample; or (iii) both (i) and (ii). In some aspects, a suitable subject has a tumor characterized by decreased expression of STAT1. In some aspects, a suitable subject has a tumor characterized by decreased expression of IFNγ. In some aspects, a suitable subject has a tumor characterized by decreased expression of STAT1 and IFNγ. In some aspects, a suitable subject has a tumor characterized by increased expression of NECTIN2. In some aspects, a suitable subject has a tumor characterized by increased expression of CSF1R. In some aspects, a suitable subject has a tumor characterized by increased expression of NECTIN2 and CSF1R. In some aspects, a suitable subject has a tumor characterized by decreased expression of STAT1 and increased expression of NECTIN2. In some aspects, a suitable subject has a tumor characterized by decreased expression of STAT1 and increased expression of NECTIN2. In some aspects, a suitable subject has a tumor characterized by decreased expression of IFNγ and increased expression of NECTIN2. In some aspects, a suitable subject has a tumor characterized by decreased expression of IFNγ and increased expression of CSF1R. In some aspects, a suitable subject has a tumor characterized by decreased expression of STAT1 and IFNγ and increased expression of NECTIN2 and CSF1R.

In certain aspects, the suitable subject, e.g., the subject having a stromal signature described herein, is to be administered and/or subsequently administered a combination therapy comprising (i) an I-O therapy described herein and (ii) one or more additional anti-cancer agents. In some aspects, the suitable subject, e.g., the subject having a stromal signature described herein, is to be administered and/or subsequently administered a combination therapy comprising (i) an anti-PD-1/PD-L1 antagonist and (ii) one or more additional anti-cancer agents. In some aspects, the suitable subject, e.g., the subject having a stromal signature described herein, is to be administered and/or subsequently administered a combination therapy comprising (i) an anti-PD-1 antibody and (ii) one or more additional anti-cancer agents. In some aspects, the suitable subject, e.g., the subject having a stromal signature described herein, is to be administered and/or subsequently administered a combination therapy comprising (i) an anti-PD-L1 antibody and (ii) one or more additional anti-cancer agents. In some aspects, the suitable subject, e.g., the subject having a stromal signature described herein, is to be administered and/or subsequently administered a combination therapy comprising (i) an anti-PD-1 antibody and (ii) an anti-CSF1R antibody. In some aspects, the suitable subject, e.g., the subject having a stromal signature described herein, is to be administered and/or subsequently administered a combination therapy comprising (i) an anti-PD-L1 antibody and (ii) an anti-CSF1R antibody.

As used herein, a suitable subject exhibits (i) an increased expression of one or more of CSF1R and NECTIN2 (“upregulated genes”) in the tumor sample compared to the expression of the one or more of CSF1R and NECTIN2 in a reference sample; (ii) a decreased expression of one or more of STAT1 and IFNγ (“down-regulated genes”) in the tumor sample compared to the expression of one or more of STAT1 and IFNγ in a reference sample; or (iii) both (i) and (ii). In some aspects, a subject is identified as being suitable for a combination therapy comprising (i) an I-O therapy, e.g., anti-PD-1/PD-L1 antagonist therapy and (ii) an additional anti-cancer therapy. As used herein a subject suitable for a combination therapy comprising (i) an I-O therapy, e.g., anti-PD-1/PD-L1 antagonist therapy has a tumor characterized by (i) decreased expression of STAT1 and/or IFNγ (“down-regulated genes”), (ii) increased expression of NECTIN2 and/or CSF1R (“upregulated genes”), or (ii) both (i) and (ii). Thus, in some aspects, the subject is identified as being suitable when the tumor sample exhibits: (i) a decreased expression of STAT1 and/or IFNγ (“down-regulated genes”) in the tumor sample compared to the expression of STAT1 and/or IFNγ in a reference sample; (ii) an increased expression of NECTIN2 and/or CSF1R (“upregulated genes”) in the tumor sample compared to the expression of NECTIN2 and/or CSF1R in a reference sample; or (iii) both (i) and (ii). In some aspects, a suitable subject has a tumor characterized by decreased expression of STAT1. In some aspects, a suitable subject has a tumor characterized by decreased expression of IFNγ. In some aspects, a suitable subject has a tumor characterized by decreased expression of STAT1 and IFNγ. In some aspects, a suitable subject has a tumor characterized by increased expression of NECTIN2. In some aspects, a suitable subject has a tumor characterized by increased expression of CSF1R. In some aspects, a suitable subject has a tumor characterized by increased expression of NECTIN2 and CSF1R. In some aspects, a suitable subject has a tumor characterized by decreased expression of STAT1 and increased expression of NECTIN2. In some aspects, a suitable subject has a tumor characterized by decreased expression of STAT1 and increased expression of NECTIN2. In some aspects, a suitable subject has a tumor characterized by decreased expression of IFNγ and increased expression of NECTIN2. In some aspects, a suitable subject has a tumor characterized by decreased expression of IFNγ and increased expression of CSF1R. In some aspects, a suitable subject has a tumor characterized by decreased expression of STAT1 and IFNγ and increased expression of NECTIN2 and CSF1R.

In certain aspects, the suitable subject is to be administered and/or subsequently administered a combination therapy comprising (i) an I-O therapy described herein and (ii) one or more additional anti-cancer agents. In some aspects, the suitable subject is to be administered and/or subsequently administered a combination therapy comprising (i) an anti-PD-1/PD-L1 antagonist and (ii) one or more additional anti-cancer agents. In some aspects, the suitable subject is to be administered and/or subsequently administered a combination therapy comprising (i) an anti-PD-1 antibody and (ii) one or more additional anti-cancer agents. In some aspects, the suitable subject is to be administered and/or subsequently administered a combination therapy comprising (i) an anti-PD-L1 antibody and (ii) one or more additional anti-cancer agents. In some aspects, the suitable subject is to be administered and/or subsequently administered a combination therapy comprising (i) an anti-PD-1 antibody and (ii) an anti-CSF1R antibody. In some aspects, the suitable subject is to be administered and/or subsequently administered a combination therapy comprising (i) an anti-PD-L1 antibody and (ii) an anti-CSF1R antibody.

In certain aspects, a combination therapy comprising (i) an anti-PD-1/PD-L1 antagonist and (ii) one or more additional anti-cancer agents is administered to a subject, wherein a tumor sample obtained from the subject exhibits: (i) a decreased expression of STAT1 and IFNγ (“down-regulated genes”) in a tumor sample obtained from the subject compared to the expression of STAT1 and IFNγ in a reference sample; (ii) an increased expression of NECTIN2 and CSF1R (“upregulated genes”) in a tumor sample obtained from the subject compared to the expression of one or more of NECTIN2 and CSF1R in a reference sample; or (iii) both (i) and (ii). In some aspects, the one or more additional anti-cancer agents comprises an anti-CSF1R antibody.

In some aspects, a combination therapy comprising (i) an anti-PD-1/PD-L1 antagonist and (ii) one or more additional anti-cancer agents is administered to a subject, wherein a tumor sample obtained from the subject does not exhibit: (i) an increased expression of STAT1 or IFNγ (“down-regulated genes”) in a tumor sample obtained from the subject compared to the expression of STAT1 or IFNγ in a reference sample; (ii) a decreased expression of NECTIN2 or CSF1R (“upregulated genes”) in a tumor sample obtained from the subject compared to the expression of one or more of NECTIN2 and CSF1R in a reference sample; or (iii) both (i) and (ii).

II.C. Antibodies

Certain aspects of the present disclosure are directed to methods of treating a suitable subject, as determined according to a method disclosed herein, using an I-O therapy. Any I-O therapy known in the art can be used in the methods described herein. In certain aspects, the I-O therapy comprises administering to the suitable subject an antibody or an antigen-binding fragment thereof the specifically binds a protein selected from PD-1, PD-L1, CTLA-4, LAG-3, TIGIT, TIM3, CSF1R, NKG2a, OX40, ICOS, CD137, KIR, TGFβ, IL-10, IL-8, IL-2, CD96, VISTA, B7-H4, Fas ligand, CXCR4, mesothelin, CD27, GITR, MICA, MICB, and any combination thereof.

In some aspects, the subject is administered a single I-O therapy, i.e., a monotherapy. In some aspects, the subject is administered an anti-PD-1 antibody monotherapy. In some aspects, the subject is administered a combination therapy comprising a first I-O therapy and a second I-O therapy. In some aspects, the subject is administered a combination therapy comprising administering a first I-O therapy and an additional anti-cancer agent. In some aspects, the additional anti-cancer agent comprises a second I-O therapy, a chemotherapy, a standard of care therapy, or any combination thereof.

In certain aspects, the subject is administered a combination therapy comprising an anti-PD-1 antibody and a second anti-cancer agent. In certain aspects, the subject is administered a combination therapy comprising an anti-PD-1 antibody and an anti-CTLA-4 antibody. In certain aspects, the subject is administered a combination therapy comprising an anti-PD-1 antibody and an anti-CSF1R antibody.

In certain aspects, the subject is administered a combination therapy comprising an anti-PD-L1 antibody and a second anti-cancer agent. In certain aspects, the subject is administered a combination therapy comprising an anti-PD-L1 antibody and an anti-CTLA-4 antibody. In certain aspects, the subject is administered a combination therapy comprising an anti-PD-L1 antibody and an anti-CSF1R antibody.

II.C.1. Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-γ production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some aspects, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one aspect, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another aspect, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain aspects, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other aspects, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one aspect, the anti-PD-1 antibody is administered at a dose of about 5 mg/kg body weight about once every 3 weeks. In another aspect, the anti-PD-1 antibody, e.g., nivolumab, is administered at a dose of about 3 mg/kg body weight about once every 2 weeks. In other aspects, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some aspects, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one aspect, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another aspects, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some aspects, the anti-PD-1 antibody is administered as a flat dose of about 200 mg at about once every 3 weeks. In other aspects, the anti-PD-1 antibody is administered as a flat dose of about 200 mg at about once every 2 weeks. In other aspects, the anti-PD-1 antibody is administered as a flat dose of about 240 mg at about once every 2 weeks. In certain aspects, the anti-PD-1 antibody is administered as a flat dose of about 480 mg at about once every 4 weeks.

In some aspects, nivolumab is administered at a flat dose of about 240 mg once about every 2 weeks. In some aspects, nivolumab is administered at a flat dose of about 240 mg once about every 3 weeks. In some aspects, nivolumab is administered at a flat dose of about 360 mg once about every 3 weeks. In some aspects, nivolumab is administered at a flat dose of about 480 mg once about every 4 weeks.

In some aspects, pembrolizumab is administered at a flat dose of about 200 mg once about every 2 weeks. In some aspects, pembrolizumab is administered at a flat dose of about 200 mg once about every 3 weeks. In some aspects, pembrolizumab is administered at a flat dose of about 400 mg once about every 4 weeks.

In some aspects, the PD-1 inhibitor is a small molecule. In some aspects, the PD-1 inhibitor comprises a millamolecule. In some aspects, the PD-1 inhibitor comprises a macrocyclic peptide. In certain aspects, the PD-1 inhibitor comprises BMS-986189. In some aspects, the PD-1 inhibitor comprises an inhibitor disclosed in International Publication No. WO2014/151634, which is incorporated by reference herein in its entirety. In some aspects, the PD-1 inhibitor comprises INCMGA00012 (Insight Pharmaceuticals). In some aspects, the PD-1 inhibitor comprises a combination of an anti-PD-1 antibody disclosed herein and a PD-1 small molecule inhibitor.

II.C.2. Anti-PD-L1 Antibodies Useful for the Disclosure

In certain aspects, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

In certain aspects, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., KO 36 (15suppl):TPS3113 (2018)), and CK-301 (Checkpoint Therapeutics; see Gorelik et al., AACR:Abstract 4606 (April 2016)).

In certain aspects, the PD-L1 antibody is atezolizumab (TECENTRIQ®). Atezolizumab is a fully humanized IgG1 monoclonal anti-PD-L1 antibody.

In certain aspects, the PD-L1 antibody is durvalumab (IMFINZI™). Durvalumab is a human IgG1 kappa monoclonal anti-PD-L1 antibody.

In certain aspects, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain aspects, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular aspect, the anti-PD-L1 antibody is durvalumab. In other aspects, the anti-PD-L1 antibody is avelumab. In some aspects, the anti-PD-L1 antibody is atezolizumab.

In some aspects, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some aspects, the anti-PD-L1 antibody is administered at a dose of about 15 mg/kg body weight at about once every 3 weeks. In other aspects, the anti-PD-L1 antibody is administered at a dose of about 10 mg/kg body weight at about once every 2 weeks.

In other aspects, the anti-PD-L1 antibody useful for the present disclosure is a flat dose. In some aspects, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some aspects, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some aspects, the anti-PD-L1 antibody is administered as a flat dose of about 1200 mg at about once every 3 weeks. In other aspects, the anti-PD-L1 antibody is administered as a flat dose of about 800 mg at about once every 2 weeks. In other aspects, the anti-PD-L1 antibody is administered as a flat dose of about 840 mg at about once every 2 weeks.

In some aspects, atezolizumab is administered as a flat dose of about 1200 mg once about every 3 weeks. In some aspects, atezolizumab is administered as a flat dose of about 800 mg once about every 2 weeks. In some aspects, atezolizumab is administered as a flat dose of about 840 mg once about every 2 weeks.

In some aspects, avelumab is administered as a flat dose of about 800 mg once about every 2 weeks.

In some aspects, durvalumab is administered at a dose of about 10 mg/kg once about every 2 weeks. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 inhibitor is a small molecule. In some aspects, the PD-L1 inhibitor comprises a millamolecule. In some aspects, the PD-L1 inhibitor comprises a macrocyclic peptide. In certain aspects, the PD-L1 inhibitor comprises BMS-986189.

In some aspects, the PD-L1 inhibitor comprises a millamolecule having a formula set forth in formula (I):

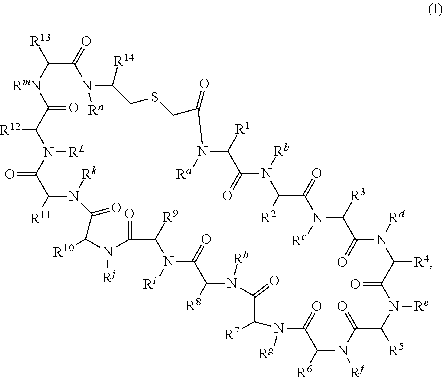

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In certain aspects the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-L1 inhibitor comprises a combination of an anti-PD-L1 antibody disclosed herein and a PD-L1 small molecule inhibitor disclosed herein.

II.C.3. Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain aspects, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular aspects, the anti-CTLA-4 antibody is ipilimumab.

In particular aspects, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular aspects, the CTLA-4 antibody is tremelimumab.

In particular aspects, the CTLA-4 antibody is MK-1308.

In particular aspects, the CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain aspects, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain aspects, the anti-CTLA-4 antibody or antigen-binding portion thereof cross-competes with ipilimumab and/or tremelimumab for binding to human CTLA-4.

In some aspects, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some aspects, the anti-CTLA-4 antibody or antigen-binding portion thereof is administered at a dose of 1 mg/kg or 3 mg/kg body weight once every 3, 4, 5, or 6 weeks. In one aspect, the anti-CTLA-4 antibody or antigen-binding portion thereof is administered at a dose of 3 mg/kg body weight once every 2 weeks. In another aspect, the anti-PD-1 antibody or antigen-binding portion thereof is administered at a dose of 1 mg/kg body weight once every 6 weeks.

In some aspects, the anti-CTLA-4 antibody or antigen-binding portion thereof is administered as a flat dose. In some aspects, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one aspect, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another aspect, the anti-CTLA-4 antibody or antigen-binding portion thereof is administered as a flat dose about once every 1, 2, 3, 4, 5, 6, 7, or 8 weeks.

In some aspects, ipilimumab is administered at a dose of about 3 mg/kg once about every 3 weeks. In some aspects, ipilimumab is administered at a dose of about 10 mg/kg once about every 3 weeks. In some aspects, ipilimumab is administered at a dose of about 10 mg/kg once about every 12 weeks. In some aspects, the ipilimumab is administered for four doses.

II.C.4. Anti-LAG-3 Antibodies

Anti-LAG-3 antibodies of the instant disclosure bind to human LAG-3. Antibodies that bind to LAG-3 have been disclosed in Int'l Publ. No. WO/2015/042246 and U.S. Publ. Nos. 2014/0093511 and 2011/0150892, each of which is incorporated by reference herein in its entirety.

An exemplary LAG-3 antibody useful in the present disclosure is 25F7 (described in U.S. Publ. No. 2011/0150892). An additional exemplary LAG-3 antibody useful in the present disclosure is BMS-986016. In one aspect, an anti-LAG-3 antibody useful for the composition cross-competes with 25F7 or BMS-986016. In another aspect, an anti-LAG-3 antibody useful for the composition binds to the same epitope as 25F7 or BMS-986016. In other aspects, an anti-LAG-3 antibody comprises six CDRs of 25F7 or BMS-986016. In another aspect, the anti-LAG-3 antibody is IMP731 (H5L7BW), MK-4280 (28G-10), REGN3767, humanized BAP050, IMP-701 (LAG-5250), TSR-033, BI754111, MGD013, or FS-118. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, and WO2014/140180, each of which is incorporated by reference herein in its entirety.

II.C.5. Anti-CD137 Antibodies

Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669, each of which is incorporated by reference herein in its entirety.

In some aspects, the anti-CD137 antibody is urelumab (BMS-663513), described in U.S. Pat. No. 7,288,638 (20H4.9-IgG4 [10C7 or BMS-663513]). In some aspects, the anti-CD137 antibody is BMS-663031 (20H4.9-IgG1), described in U.S. Pat. No. 7,288,638. In some aspects, the anti-CD137 antibody is 4E9 or BMS-554271, described in U.S. Pat. No. 6,887,673. In some aspects, the anti-CD137 antibody is an antibody disclosed in U.S. Pat. Nos. 7,214,493; 6,303,121; 6,569,997; 6,905,685; or 6,355,476. In some aspects, the anti-CD137 antibody is 1D8 or BMS-469492; 3H3 or BMS-469497; or 3E1, described in U.S. Pat. No. 6,362,325. In some aspects, the anti-CD137 antibody is an antibody disclosed in issued U.S. Pat. No. 6,974,863 (such as 53A2). In some aspects, the anti-CD137 antibody is an antibody disclosed in issued U.S. Pat. No. 6,210,669 (such as 1D8, 3B8, or 3E1). In some aspects, the antibody is Pfizer's PF-05082566 (PF-2566). In other aspects, an anti-CD137 antibody useful for the methods disclosed herein cross-competes with the anti-CD137 antibodies disclosed herein. In some aspects, an anti-CD137 antibody binds to the same epitope as the anti-CD137 antibody disclosed herein. In other aspects, an anti-CD137 antibody useful in the disclosure comprises six CDRs of the anti-CD137 antibodies disclosed herein.

II.C.6. Anti-MR Antibodies

Antibodies that bind specifically to KIR block the interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448, each of which is incorporated by reference herein in its entirety.

One anti-KIR antibody useful in the present disclosure is lirilumab (also referred to as BMS-986015, IPH2102, or the S241P variant of 1-7F9), first described in Int'l Publ. No. WO 2008/084106. An additional anti-KIR antibody useful in the present disclosure is 1-7F9 (also referred to as IPH2101), described in Int'l Publ. No. WO 2006/003179. In one aspect, an anti-KIR antibody for the present composition cross competes for binding to KIR with lirilumab or I-7F9. In another aspect, an anti-KIR antibody binds to the same epitope as lirilumab or I-7F9. In other aspects, an anti-KIR antibody comprises six CDRs of lirilumab or I-7F9.

II.C.7. Anti-GITR Antibodies

Anti-GITR antibodies useful in the methods disclosed herein include any anti-GITR antibody that binds specifically to human GITR target and activates the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841, each of which is incorporated by reference herein in its entirety.

In one aspect, an anti-GITR antibody useful in the present disclosure is TRX518 (described in, for example, Schaer et al. Curr Opin Immunol. (2012) April; 24(2): 217-224, and WO/2006/105021). In another aspect, the anti-GITR antibody is selected from MK4166, MK1248, and antibodies described in WO11/028683 and U.S. Pat. No. 8,709,424, and comprising, e.g., a VH chain comprising SEQ ID NO: 104 and a VL chain comprising SEQ ID NO: 105 (wherein the SEQ ID NOs are from WO11/028683 or U.S. Pat. No. 8,709,424). In certain aspects, an anti-GITR antibody is an anti-GITR antibody that is disclosed in WO2015/031667, e.g., an antibody comprising VH CDRs 1-3 comprising SEQ ID NOs: 31, 71 and 63 of WO2015/031667, respectively, and VL CDRs 1-3 comprising SEQ ID NOs: 5, 14 and 30 of WO2015/031667. In certain aspects, an anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979). In certain aspects, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain aspects, an anti-GITR antibody is an anti-GITR antibody that is described in PCT/US2015/033991 (e.g., an antibody comprising the variable regions of 28F3, 18E10 or 19D3).

In certain aspects, the anti-GITR antibody cross-competes with an anti-GITR antibody described herein, e.g., TRX518, MK4166 or an antibody comprising a VH domain and a VL domain amino acid sequence described herein. In some aspects, the anti-GITR antibody binds the same epitope as that of an anti-GITR antibody described herein, e.g., TRX518 or MK4166. In certain aspects, the anti-GITR antibody comprises the six CDRs of TRX518 or MK4166.

II.C.8. Anti-TIM3 Antibodies

Any anti-TIM3 antibody or antigen binding fragment thereof known in the art can be used in the methods described herein. In some aspects, the anti-TIM3 antibody is be selected from the anti-TIM3 antibodies disclosed in Int'l Publ. Nos.WO2018013818, WO/2015/117002 (e.g., MGB453, Novartis), WO/2016/161270 (e.g., TSR-022, Tesaro/AnaptysBio), WO2011155607, WO2016/144803 (e.g., STI-600, Sorrento Therapeutics), WO2016/071448, WO17055399; WO17055404, WO17178493, WO18036561, WO18039020 (e.g., Ly-3221367, Eli Lilly), WO2017205721, WO17079112; WO17079115; WO17079116, WO11159877, WO13006490, WO2016068802 WO2016068803, WO2016/111947, and WO/2017/031242, each of which is incorporated by reference herein in its entirety.

II.C.9. Anti-OX40 Antibodies

Any antibody or antigen-binding fragment thereof that specifically binds OX40 (also known as CD134, TNFRSF4, ACT35 and/or TXGP1L) can be used in the methods disclosed herein. In some aspects, the anti-OX40 antibody is BMS-986178 (Bristol-Myers Squibb Company), described in Int'l Publ. No. WO20160196228. In some aspects, the anti-OX40 antibody is selected from the anti-OX40 antibodies described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182, each of which is incorporated by reference herein in its entirety.

II.C.10. Anti-NKG2A Antibodies

Any antibody or antigen-binding fragment thereof that specifically binds NKG2A can be used in the methods disclosed herein. NKG2A is a member of the C-type lectin receptor family that is expressed on natural killer (NK) cells and a subset of T lymphocytes. Specifically, NKG2A primarily expressed on tumor infiltrating innate immune effector NK cells, as well as on some CD8+ T cells. Its natural ligand human leukocyte antigen E (HLA-E) is expressed on solid and hematologic tumors. NKG2A is an inhibitory receptor that blinds HLA-E.

In some aspects, the anti-NKG2A antibody may be BMS-986315, a human monoclonal antibody that blocks the interaction of NKG2A to its ligand HLA-E, thus allowing activation of an anti-tumor immune response. In some aspects, the anti-NKG2A antibody is a checkpoint inhibitor that activates T cells, NK cells, and/or tumor-infiltrating immune cells. In some aspects, the anti-NKG2A antibody is selected from the anti-NKG2A antibodies described in, for example, WO 2006/070286 (Innate Pharma S.A.; University of Genova); U.S. Pat. No. 8,993,319 (Innate Pharma S.A.; University of Genova); WO 2007/042573 (Innate Pharma S/A; Novo Nordisk A/S; University of Genova); U.S. Pat. No. 9,447,185 (Innate Pharma S/A; Novo Nordisk A/S; University of Genova); WO 2008/009545 (Novo Nordisk A/S); U.S. Pat. Nos. 8,206,709; 8,901,283; 9,683,041 (Novo Nordisk A/S); WO 2009/092805 (Novo Nordisk A/S); U.S. Pat. Nos. 8,796,427 and 9,422,368 (Novo Nordisk A/S); WO 2016/134371 (Ohio State Innovation Foundation); WO 2016/032334 (Janssen); WO 2016/041947 (Innate); WO 2016/041945 (Academisch Ziekenhuis Leiden H.O.D.N. LUMC); WO 2016/041947 (Innate Pharma); and WO 2016/041945 (Innate Pharma), each of which is incorporated by reference herein in its entirety.

II.C.11. Anti-ICOS Antibodies

Any antibody or antigen-binding fragment thereof that specifically binds ICOS can be used in the methods disclosed herein. ICOS is an immune checkpoint protein that is a member of the CD28-superfamily. ICOS is a 55-60 kDa type I transmembrane protein that is expressed on T cells after T cell activation and co-stimulates T-cell activation after binding its ligand, ICOS-L (B7H2). ICOS is also known as inducible T-cell co-stimulator, CVID1, AILIM, inducible costimulator, CD278, activation-inducible lymphocyte immunomediatory molecule, and CD278 antigen.

In some aspects, the anti-ICOS antibody is BMS-986226, a humanized IgG monoclonal antibody that binds to and stimulates human ICOS. In some aspects, the anti-ICOS antibody is selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited), each of which is incorporated by reference herein in its entirety.

II.C.12. Anti-TIGIT Antibodies

Any antibody or antigen-binding fragment thereof that specifically binds TIGIT can be used in the methods disclosed herein. In some aspects, the anti-TIGIT antibody is BMS-986207. In some aspects, the anti-TIGIT antibody is clone 22G2, as described in WO 2016/106302. In some aspects, the anti-TIGIT antibody is MTIG7192A/RG6058/R07092284, or clone 4.1D3, as described in WO 2017/053748. In some aspects, the anti-TIGIT antibody is selected from the anti-TIGIT antibodies described in, for example, WO 2016/106302 (Bristol-Myers Squibb Company) and WO 2017/053748 (Genentech).

II.C.13. Anti-CSF1R Antibodies

Any antibody or antigen-binding fragment thereof that specifically binds CSF1R can be used in the methods disclosed herein. In some aspects, the anti-CSF1R antibody is an antibody species disclosed in any of international publications WO2013/132044, WO2009/026303, WO2011/140249, or WO2009/112245, such as cabiralizumab, RG7155 (emactuzumab), AMG820, SNDX 6352 (UCB 6352), CXIIG6, IMC-CS4, JNJ-40346527, MCS110, or the anti-CSF1R antibody in the methods is replaced with an anti-CSF1R inhibitor or anti-CSF1 inhibitor such as BLZ-945, pexidartinib (PLX3397, PLX108-01), AC-708, PLX-5622, PLX7486, ARRY-382, or PLX-73086.

II.D. Additional Anti-Cancer Therapies

In some aspects of the present disclosure, the methods disclosed herein further comprise administering an I-O therapy, e.g., an anti-PD-1 antibody or an anti-PD-L1 antibody, and one or more additional anti-cancer therapies. In certain aspects, the method comprising administering (i) a first I-O therapy, e.g., an anti-PD-1 antibody or an anti-PD-L1 antibody), (ii) a second I-O therapy, e.g., an anti-CTLA-4 antibody or an anti-CSF1R antibody, and (iii) one or more additional anti-cancer therapies.

The additional anti-cancer therapy can comprise any therapy known in the art for the treatment of a tumor in a subject and/or any standard-of-care therapy, as disclosed herein. In some aspects, the additional anti-cancer therapy comprises a surgery, a radiation therapy, a chemotherapy, an immunotherapy, or any combination thereof. In some aspects, the additional anti-cancer therapy comprises a chemotherapy, including any chemotherapy disclosed herein.

Any chemotherapy known in the art can be used in the methods disclosed herein. In some aspects, the chemotherapy is a platinum based-chemotherapy. Platinum-based chemotherapies are coordination complexes of platinum. In some aspects, the platinum-based chemotherapy is a platinum-doublet chemotherapy. In some aspects, the chemotherapy is administered at the approved dose for the particular indication. In other aspects, the chemotherapy is administered at any dose disclosed herein. In some aspects, the platinum-based chemotherapy is cisplatin, carboplatin, oxaliplatin, satraplatin, picoplatin, Nedaplatin, Triplatin, Lipoplatin, or combinations thereof. In certain aspects, the platinum-based chemotherapy is any other platinum-based chemotherapy known in the art. In some aspects, the chemotherapy is the nucleotide analog gemcitabine. In an aspect, the chemotherapy is a folate antimetabolite. In an aspect, the folate antimetabolite is pemetrexed. In certain aspects the chemotherapy is a taxane. In other aspects, the taxane is paclitaxel. In some aspects, the chemotherapy is any other chemotherapy known in the art. In certain aspects, at least one, at least two or more chemotherapeutic agents are administered in combination with the I-O therapy. In some aspects, the I-O therapy is administered in combination with gemcitabine and cisplatin. In some aspects, the I-O therapy is administered in combination with pemetrexed and cisplatin. In certain aspects, the I-O therapy is administered in combination with gemcitabine and pemetrexed. In one aspect, the I-O therapy is administered in combination with paclitaxel and carboplatin. In an aspect, an I-O therapy is additionally administered.

In some aspects, the additional anti-cancer therapy comprises an immunotherapy. In some aspects, the additional anti-cancer therapy comprises administration of an antibody or antigen-binding portion thereof that specifically binds LAG-3, TIGIT, TIM3, NKG2a, CSF1R, OX40, ICOS, MICA, MICB, CD137, KIR, TGFβ, IL-10, IL-8, B7-H4, Fas ligand, CXCR4, mesothelin, CD27, GITR, or any combination thereof.

II.E. Tumors

In some aspects, the tumor is derived from a cancer selected from the group consisting of hepatocellular cancer, gastroesophageal cancer, melanoma, bladder cancer, lung cancer, kidney cancer, head and neck cancer, colon cancer, and any combination thereof. In certain aspects, the tumor is derived from a hepatocellular cancer, wherein the tumor has a high inflammatory signature score. In certain aspects, the tumor is derived from a gastroesophageal cancer, wherein the tumor has a high inflammatory signature score. In certain aspects, the tumor is derived from a melanoma, wherein the tumor has a high inflammatory signature score. In certain aspects, the tumor is derived from a bladder cancer, wherein the tumor has a high inflammatory signature score. In certain aspects, the tumor is derived from a lung cancer, wherein the tumor has a high inflammatory signature score. In certain aspects, the tumor is derived from a kidney cancer, wherein the tumor has a high inflammatory signature score. In certain aspects, the tumor is derived from a head and neck cancer, wherein the tumor has a high inflammatory signature score. In certain aspects, the tumor is derived from a colon cancer, wherein the tumor has a high inflammatory signature score.

In certain aspects, the subject has received one, two, three, four, five or more prior cancer treatments. In other aspects, the subject is treatment-naïve. In some aspects, the subject has progressed on other cancer treatments. In certain aspects, the prior cancer treatment comprised an immunotherapy. In other aspects, the prior cancer treatment comprised a chemotherapy. In some aspects, the tumor has reoccurred. In some aspects, the tumor is metastatic. In other aspects, the tumor is not metastatic. In some aspects, the tumor is locally advanced.

In some aspects, the subject has received a prior therapy to treat the tumor and the tumor is relapsed or refractory. In certain aspects, the at least one prior therapy comprises a standard-of-care therapy. In some aspects, the at least one prior therapy comprises a surgery, a radiation therapy, a chemotherapy, an immunotherapy, or any combination thereof. In some aspects, the at least one prior therapy comprises a chemotherapy. In some aspects, the subject has received a prior immuno-oncology (I-O) therapy to treat the tumor and the tumor is relapsed or refractory. In some aspects, the subject has received more than one prior therapy to treat the tumor and the subject is relapsed or refractory. In other aspects, the subject has received either an anti-PD-1 or anti-PD-L1 antibody therapy.

In some aspects, the previous line of therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In one particular aspect, the platinum-based therapy comprises carboplatin.

In some aspects, the at least one prior therapy is selected from a therapy comprising administration of an anti-cancer agent selected from the group consisting of a platinum agent (e.g., cisplatin, carboplatin), a taxanes agent (e.g., paclitaxel, albumin-bound paclitaxel, docetaxel), vinorelbine, vinblastine, etoposide, pemetrexed, gemcitabine, bevacizumab (AVASTIN®), erlotinib (TARCEVA®), crizotinib (XALKORI®), cetuximab (ERBITUX®), and any combination thereof. In certain aspects, the at least one prior therapy comprises a platinum-based doublet chemotherapy.

In some aspects, the subject has experienced disease progression after the at least one prior therapy. In certain aspects, the subject has received at least two prior therapies, at least three prior therapies, at least four prior therapies, or at least five prior therapies. In certain aspects, the subject has received at least two prior therapies. In one aspect, the subject has experienced disease progression after the at least two prior therapies. In certain aspects, the at least two prior therapies comprises a first prior therapy and a second prior therapy, wherein the subject has experienced disease progression after the first prior therapy and/or the second prior therapy, and wherein the first prior therapy comprises a surgery, a radiation therapy, a chemotherapy, an immunotherapy, or any combination thereof; and wherein the second prior therapy comprises a surgery, a radiation therapy, a chemotherapy, an immunotherapy, or any combination thereof. In some aspects, the first prior therapy comprises a platinum-based doublet chemotherapy, and the second prior therapy comprises a single-agent chemotherapy. In certain aspects, the single-agent chemotherapy comprises docetaxel.

II.F. Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some aspects, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH2O), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. Therefore, in some aspects, the pharmaceutical composition for the present disclosure can further comprise recombinant human hyaluronidase enzyme, e.g., rHuPH20.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al, 2013; Rini et al., 2011) support the selection of a nivolumab dose lower than 10 mg/kg.

Treatment is continued as long as clinical benefit is observed or until unacceptable toxicity or disease progression occurs. Nevertheless, in certain aspects, the antibodies disclosed herein are administered at doses that are significantly lower than the approved dosage, i.e., a subtherapeutic dosage, of the agent. The antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### III. Kits

Also within the scope of the present disclosure are kits comprising (a) an anti-PD-1 antibody or an anti-PD-L1 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from 0.1 to 10 mg/kg body weight of an anti-PD-1 antibody or a dosage ranging from 0.1 to 20 mg/kg body weight of an anti-PD-L1 antibody; and (b) instructions for using the anti-PD-1 antibody or the anti-PD-L1 antibody in the methods disclosed herein. This disclosure further provides a kit for treating a subject afflicted with a tumor, the kit comprising: (a) a dosage ranging from about 4 mg to about 500 mg of an anti-PD-1 antibody or a dosage ranging from about 4 mg to about 2000 mg of an anti-PD-L1 antibody; and (b) instructions for using the anti-PD-1 antibody or the anti-PD-L1 antibody in the methods disclosed herein. In some aspects, this disclosure provides a kit for treating a subject afflicted with a tumor, the kit comprising: (a) a dosage ranging from 200 mg to 800 mg of an anti-PD-1 antibody or a dosage ranging from 200 mg to 1800 mg of an anti-PD-L1 antibody; and (b) instructions for using the anti-PD-1 antibody or the anti-PD-L1 antibody in the methods disclosed herein.

In certain aspects for treating human patients, the kit comprises an anti-human PD-1 antibody disclosed herein, e.g., nivolumab or pembrolizumab. In certain aspects for treating human patients, the kit comprises an anti-human PD-L1 antibody disclosed herein, e.g., atezolizumab, durvalumab, or avelumab.

In some aspects, the kit further comprises an anti-CTLA-4 antibody. In certain aspects for treating human patients, the kit comprises an anti-human CTLA-4 antibody disclosed herein, e.g., ipilimumab, tremelimumab, MK-1308, or AGEN-1884.

In some aspects, the kit further includes a gene panel assay disclosed herein. In some aspects, the kit further includes instructions to administer the anti-PD-1 antibody or the anti-PD-L1 antibody to a suitable subject according to the methods disclosed herein.

All of the references cited above, as well as all references cited herein, are incorporated herein by reference in their entireties.

The following examples are offered by way of illustration and not by way of limitation.

### EXAMPLES

### Example 1

Inflammation of the tumor microenvironment (TME), marked by infiltration of CD8+ T cells, has been associated with improved clinical outcomes across multiple tumor types 1. Parenchymal infiltration of CD8+ T cells has been associated with improved survival with immuno-oncology (I-O) treatment, and intratumoral localization also affects outcome, highlighting the importance of spatial analysis of CD8+ T cells within the TME. CD8+ T-cell patterns within tumors, as assessed by immunohistochemistry (IHC), are variable and may be classified as: (i) immune desert (minimal T-cell infiltrate); (ii) immune excluded (T cells confined to tumor stroma or invasive margin); or (iii) Immune inflamed (T cells infiltrating tumor parenchyma, positioned in proximity to tumor cells).

Emerging data suggest that artificial intelligence (AI)-based image analysis can be used to characterize the tumor parenchymal and stromal compartments in the TME. Pathology data can be quantified, and IHC assays are amenable to future in vitro diagnostic development; however, there is limited capacity for multiplexing within IHC assays.

Gene expression profiling (GEP) provides an alternative approach for assessment of inflammation in the TME. Associations between inflammatory gene signature scores and response to I-O therapy have been demonstrated in multiple tumor types.

An inflammation gene panel comprising 95 genes for GEP, in combination with AI-based image analysis, was used to analyze patterns of T-cell infiltration in tumor parenchymal and stromal compartments and to evaluate potential biomarker assays for I-O therapy.

Objectives

The first objection of the present study is to quantify regional CD8+ T-cell localization using AI-based image analysis. The second objective is to identify gene signatures that define CD8+ T-cell infiltration and localization to parenchymal and stromal compartments in the TME.

Methods

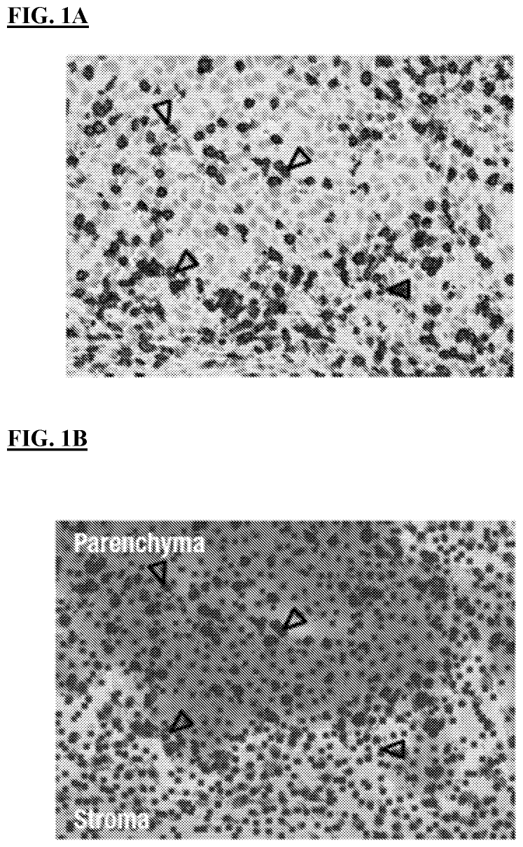

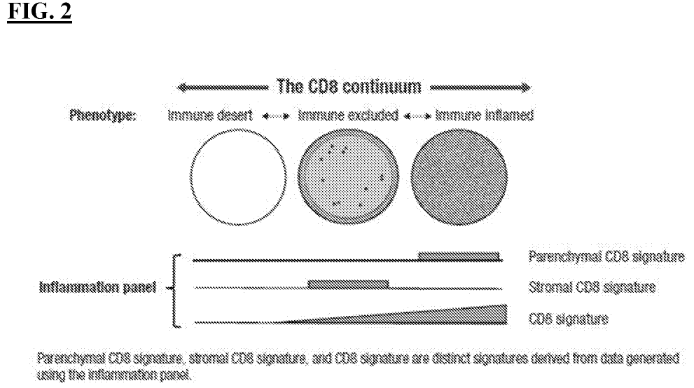

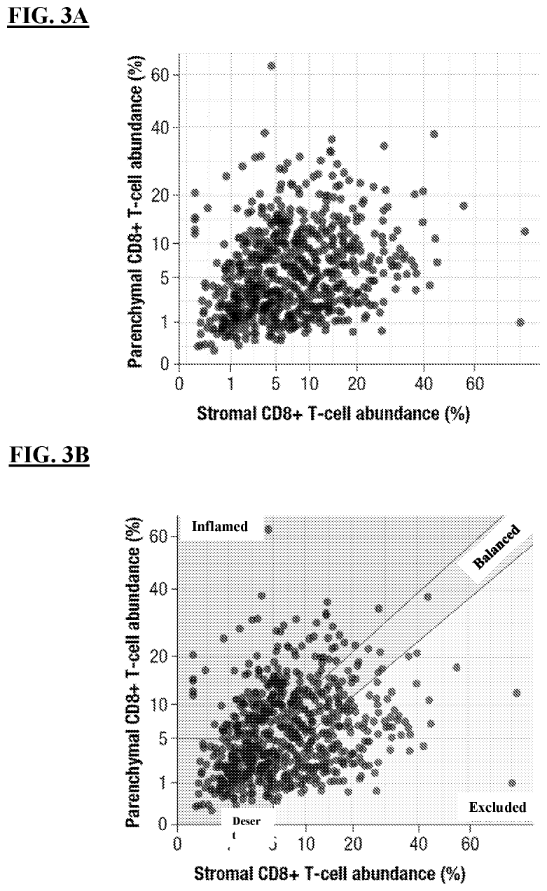

IHC for CD8 expression (CD8 IHC) and GEP were performed on commercially procured melanoma (n=158) and squamous cell carcinoma of the head and neck (SCCHN; n=250) tumor samples. CD8 IHC was performed by Mosaic Laboratories using a monoclonal CD8 (clone C8/144B) antibody (Dako, an Agilent Technologies Co, Santa Clara, Calif.). A convolutional neural network (PathAI Inc, Boston, Mass.) and AI-based image analysis algorithms were used to quantify the abundance of CD8+ T cells in tumor parenchymal and stromal regions (FIGS. 1A-1B; Table 1)

TABLE 1
AI-based quantification of CD8+ T cells in Tumor
Parenchymal and Stromal Regions

| Computed Statistic | Count |
|---|---|
| CD8+ cells | 48,819 |
| CD8+ cells in tumor | 48,439 |
| Total cells | 195,487 |
| Total cells in tumor | 193,455 |
| Tumor Area = 31.12 mm² | |

GEP was performed by next-generation sequencing (NGS) using an inflammation panel. The inflammation panel comprises 95 genes, including genes related to tumor inflammation and other I-O processes, housekeeping genes, and control genes. This inflammation panel measures mRNA expression levels of all 95 genes on the panel.

Generalized constrained regression models were used to predict CD8+ T-cell abundance in the tumor parenchyma and stroma using specific gene signatures (FIG. 2). Repeated cross-validation (100 repeats of 5-fold cross-validation) was performed to estimate generalization of the models.

Adjusted coefficient of determination (R2) was used to analyze the relationship between CD8+ T-cell abundance and the tumor parenchymal and stromal CD8 signatures.

Results

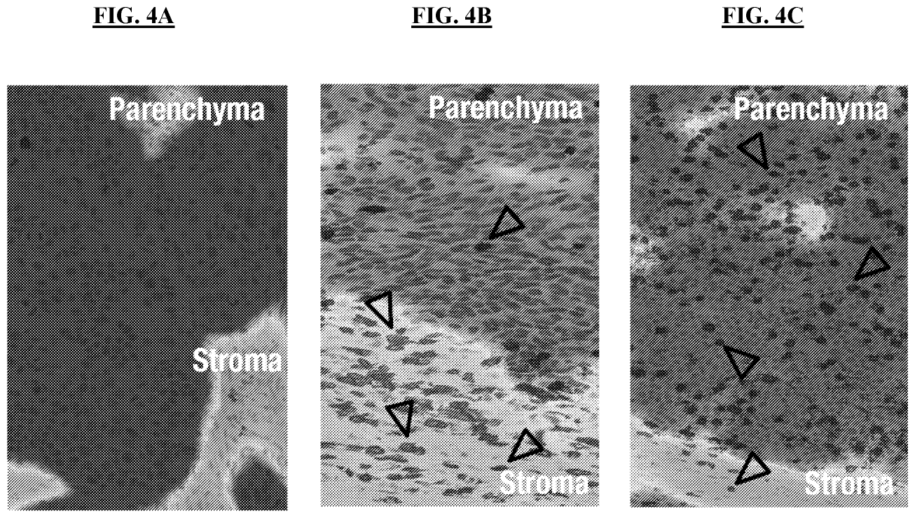

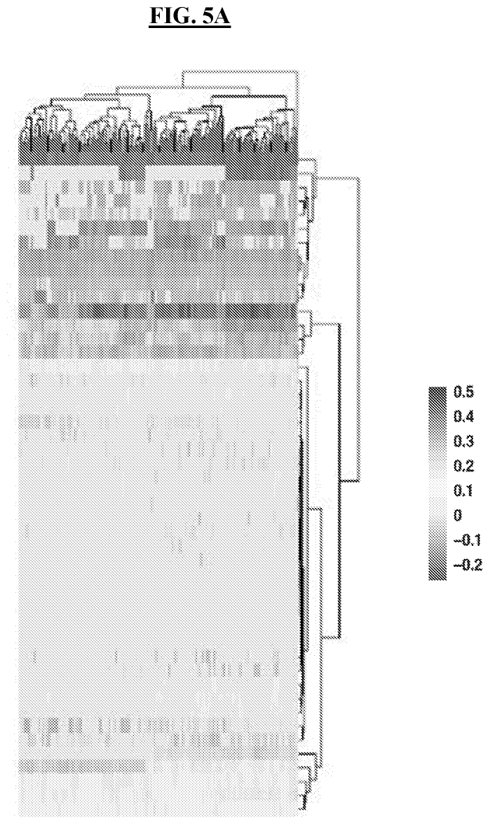

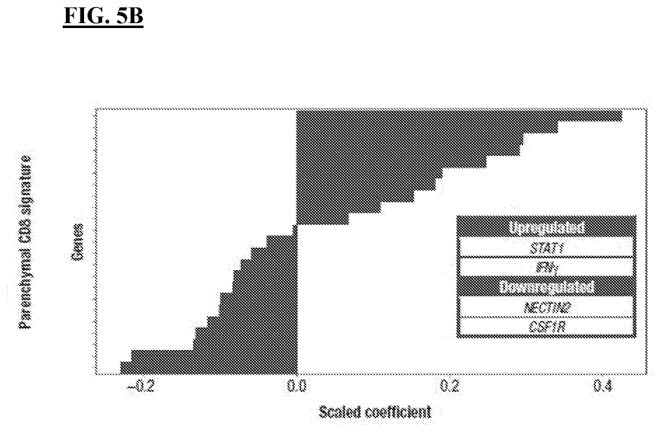

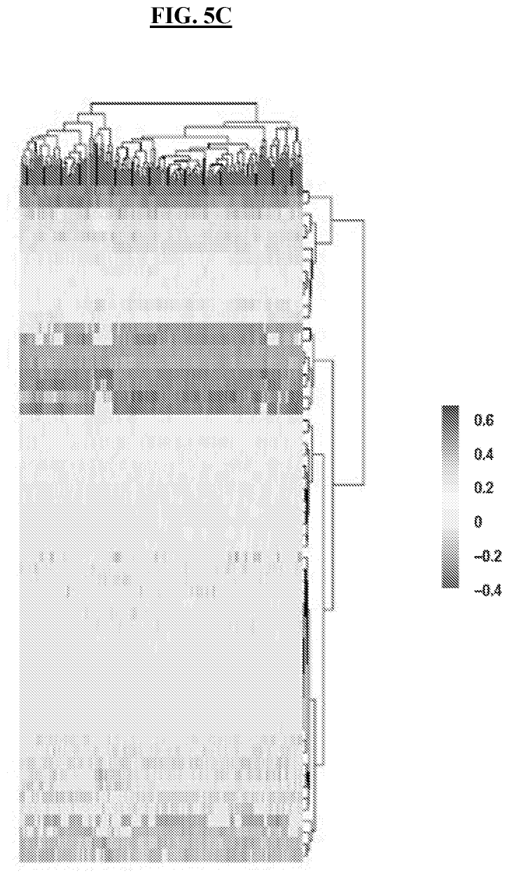

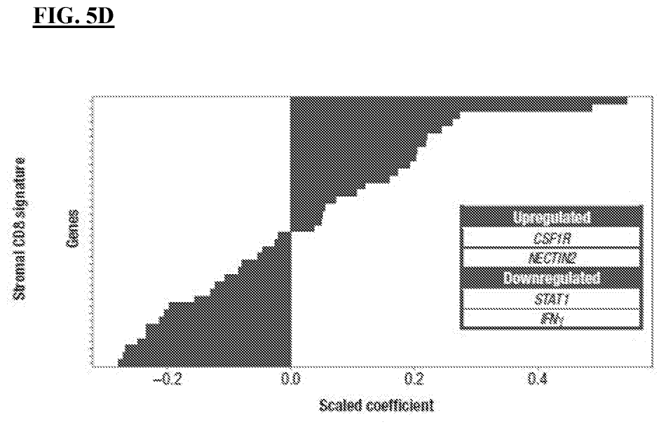

Immune phenotypes of melanoma and SCCHN samples were defined and characterized using AI-based quantification of CD8+ T cells (FIGS. 1B and 4A-4C). Using the inflammation panel to analyze melanoma and SCCHN samples, gene signatures that correlated with parenchymal and stromal CD8+ T-cell abundance were derived, demonstrating that different gene signatures can be developed using the same assay (FIGS. 2 and 5A-5D).

A parenchymal CD8 gene signature correlated with tumor parenchymal CD8+ T-cell abundance (FIGS. 5A-5B). Of the 23 genes in the signature, 10 were upregulated and 13 were downregulated; top predictors of parenchymal CD8+ T-cell abundance included: upregulation of STAT1 and IFNγ, and downregulation of NECTIN2 and CSF1R (FIG. 5B).

A stromal CD8 gene signature correlated with tumor stromal CD8+ T-cell abundance (FIGS. 5C-5D). Of the 38 genes in the signature, 19 were upregulated and 19 were downregulated. Top predictors of tumor stromal CD8+ T-cell abundance included: upregulation of CSF1R and NECTIN2, and downregulation of STAT1 and IFNγ.

Limited overlap was observed between the 2 sets of genes. Some genes that correlated with stromal CD8+ T-cell localization showed inverse correlation with parenchymal CD8+ T-cell localization (FIGS. 5A-5D). Upregulation of STAT1 and IFNγ correlated with parenchymal CD8+ T-cell abundance, whereas downregulation of these genes correlated with stromal CD8+ T-cell abundance. Downregulation of CSF1R and NECTIN2 correlated with parenchymal CD8+ T-cell abundance, whereas upregulation of these genes correlated with stromal CD8+ T-cell abundance.

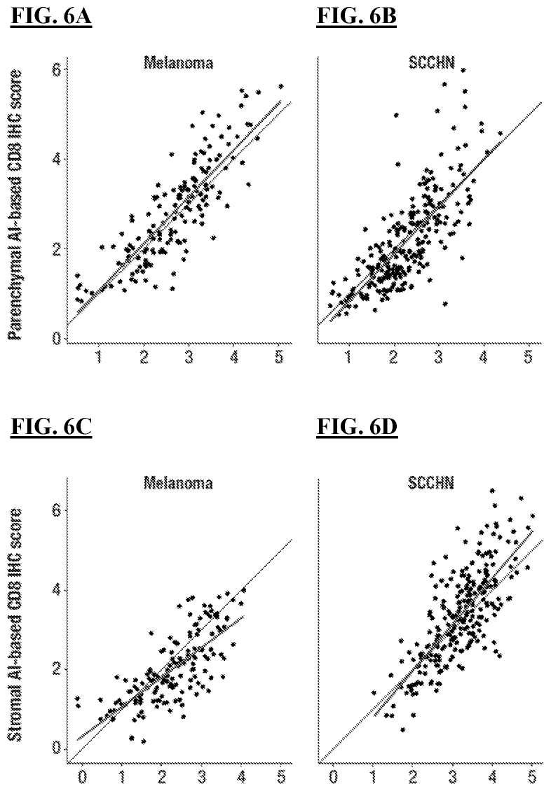

Gene signature scores were developed from the tumor parenchymal and stromal CD8 signatures. For both tumor-specific and pooled analyses of melanoma and SCCHN samples, gene signature scores were highly concordant with AI-based quantification of CD8+ T cells, depending on regional CD8+ T-cell infiltration (FIGS. 6A-6D). Adjusted R2 for parenchymal CD8 signature score (pooled analysis)=0.67 (P<0.01). Adjusted R2 for stromal CD8 signature score (pooled analysis)=0.65 (P<0.01).

The present example describes a GEP-based, investigational inflammation assay with the potential to be utilized prospectively in a clinical trial setting. Using this panel, gene signatures were further derived that predict CD8+ T-cell abundance in the tumor parenchyma and stroma. Parenchymal and stromal CD8 signature scores were concordant with CD8+ T-cell abundance determined by IHC in melanoma and SCCHN samples (individually and pooled), indicating that these gene signatures may be utilized to assess T-cell abundance. Combining GEP with AI-based image analysis could be developed as an analytical tool to characterize immune cell infiltration in the TME.

### Example 2

The association of gene expression signatures of CD8+ T-cell infiltration (CD8 signature, CD8 topology signatures) and CD8 IHC with EMT gene expression (CD8.IHC_EMT) with response to nivolumab was compared in patients with urothelial cancer (UC).

### Methods

Patients

Patients with platinum-pretreated metastatic UC received nivolumab in a clinical trial (NCT02387996), and objective response (OR) was assessed by blinded independent central review.

In evaluable baseline samples, CD8 IHC was performed using monoclonal anti-CD8 (C8/144B) by Mosaic Laboratories (Lake Forest, Calif.); CD8+ T-cell infiltration in parenchymal and stromal areas was quantified, and tumors were defined as immune-desert, immune excluded, or immune-inflamed phenotypes (FIGS. 4A-4C). EMT gene expression was measured using the HTG EdgeSeq Biomarker Panels (HTG Molecular Diagnostics, Tucson, Ariz.), and an EMT signature score was calculated by the arithmetic mean of the EMT gene expression levels.

GEP by next-generation sequencing using an inflammation panel was performed on evaluable UC samples from patients. Scores were derived from previously identified gene expression signatures that assess CD8+ T-cell abundance (CD8 signature) and localization to tumor parenchyma and stroma (CD8 topology signatures).

Biomarker Models and Statistical Analysis

Biomarker models were developed that included single signatures and the multiplicative interactions among 2 or 3 gene signatures (Table 2) The dual and triple CD8 signatures evaluated consider CD8+ T-cell inflammation in the TME as well as in specific tumor regions within the TME.

TABLE 2
Biomarker models evaluated for association with outcomes
in patients with UC treated with nivolumab

| Biomarker | | | |
|---|---|---|---|
| Model | Variable 1 | Variable 2 | Variable 3 |
| CD8 Signature | CD8 Signature | Null | Null |
| Parenchymal | Parenchymal | Null | Null |
| CD8 Signature | CD8 Signature | | |
| Dual CD8 | CD8 Signature | Parenchymal | Null |
| Signature | | CD8 Signature | |
| Triple CD8 | CD8 Signature | Parenchymal | Stromal CD8 |
| Signature | | CD8 Signature | Signature |
| CD8.IHC_EMT | CD8 IHC | EMT Signature | Null |

Cox proportional hazards regression models were used to assess the dependence of progression-free survival (PFS) or overall survival (OS) on the biomarker scores Hazard ratios (HRs) and two-sided 95% confidence intervals (CIs; calculated based on Wald test statistics) represent the difference between the 75th and 25th biomarker percentiles; graphs were scaled to compare log 2(HR) values. Kaplan-Meier plots based on categorization of the biomarker scores by tertile (high, medium, low) were used to illustrate associations with PFS and OS. Logistic regression models were used to assess the dependence of OR on the biomarker scores HRs and two-sided 95% CIs (calculated based on Wald test statistics) represent the difference between the 75th and 25th biomarker percentiles; graphs were scaled to compare log 2(HR) values. Receiver operating characteristic (ROC) curves and area under the ROC curves (AUC) were used to evaluate the performance of the models as predictors of OR.

### Results

GEP-derived CD8 topology signatures and CD8.IHC_EMT were evaluable in 205 of 270 (76%) and 187 of 270 (69%) patients, respectively, in the clinical trial. Baseline characteristics and clinical outcomes were similar in the cohorts to the overall study population (Table 3).

TABLE 3
Baseline characteristics and outcomes for overall study
population and biomarker cohorts

| Baseline | | | CD8.IHC_EMT |
|---|---|---|---|
| Characteristic | NCT02387996 | GEP Evaluable^{a} | Evaluable^{a} |
| Patients, N | | | |
| Mean age, years | 65.0 (38, 90) | 65.2 (38, 90) | 65.4 (40, 90) |
| (min, max) | | | |
| OR, %: | 20.4 | 20 | 18.7 |
| responders | | | |
| (CR + PR) | | | |
| OR, %: | 79.6 | 80 | 81.3 |
| nonresponders | | | |
| (PD + SD + NE) | | | |
| Median PFS, | 1.94 (1.87-2.33) | 1.90 (1.87-2.56) | 1.91 (1.84-2.46) |
| months | | | |
| (95% CI) | | | |
| Median OS, | 8.57 (6.04-11.27) | 9.49 (6.31-11.40) | 9.10 (6.11-11.30) |
| months | | | |
| (95% CI) | | | |

^{a}Reasons for samples not evaluable for GEP and CD8.IHC_EMT assessment included insufficient tumor biopsy material or insufficient RNA quality.
CR, complete response;
NE, nonevaluable;
PD, progressive disease;
PR, partial response;
SD, stable disease.

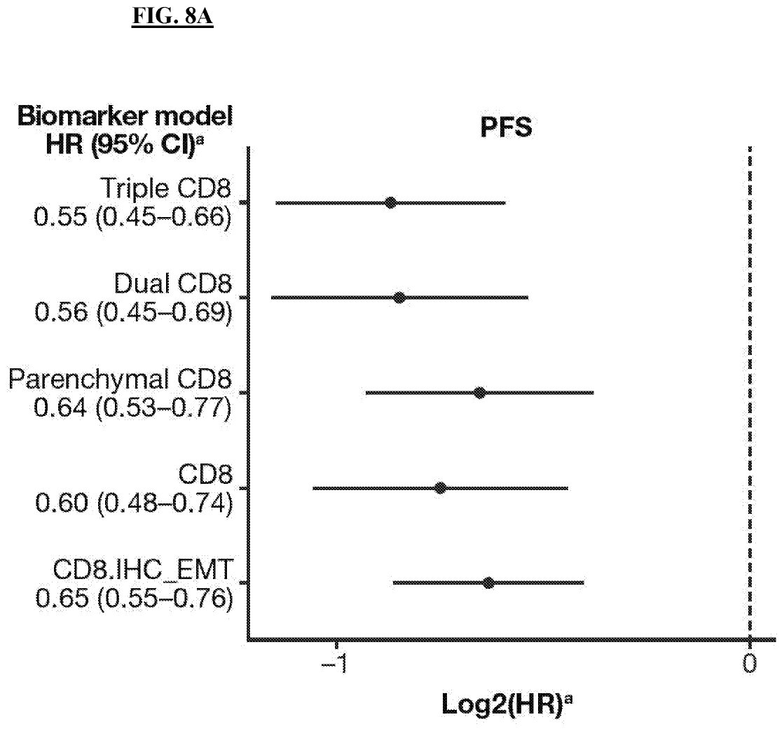

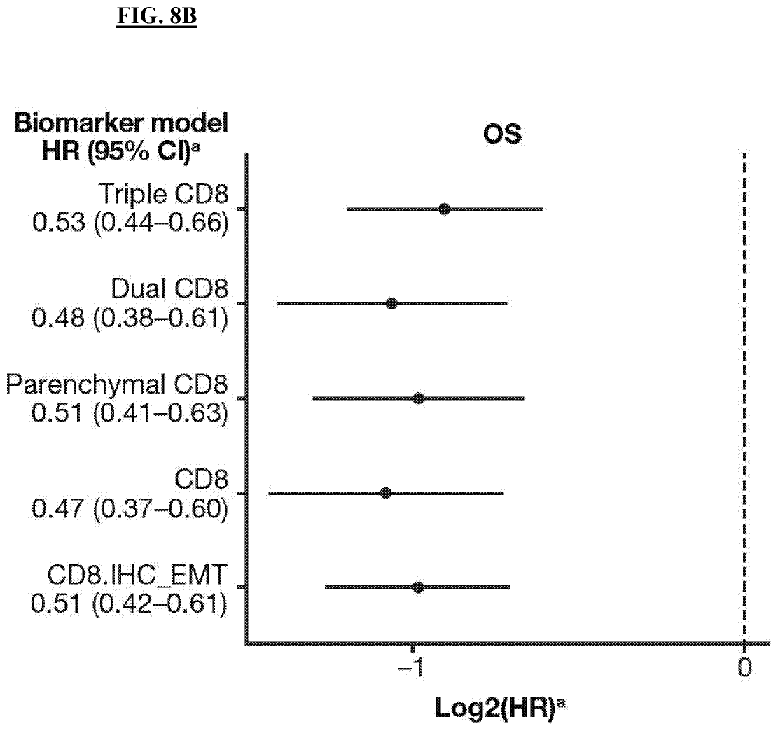

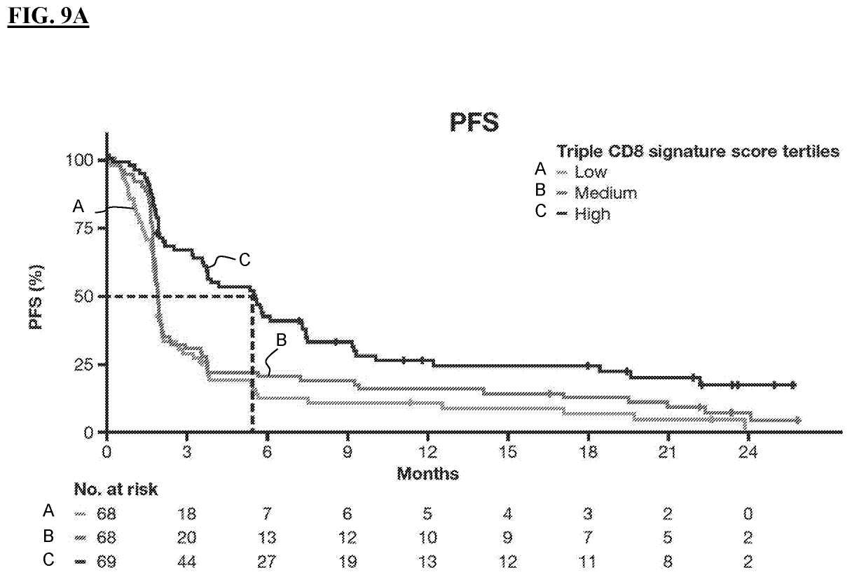

The CD8 signature biomarker models (CD8, parenchymal CD8, dual CD8, and triple CD8) showed similar associations with PFS and OS as CD8.IHC_EMT HRs for PFS ranged from 0.55 (95% CI, 0.45-0.66) for triple CD8 to 0.65 (95% CI, 0.55-0.76) for the CD8.IHC_EMT signature (FIG. 8A). HRs for OS ranged from 0.47 for the CD8 signature (95% CI, 0.37-0.60) to 0.53 (95% CI, 0.44-0.66) for the triple CD8 signature (FIG. 8B)

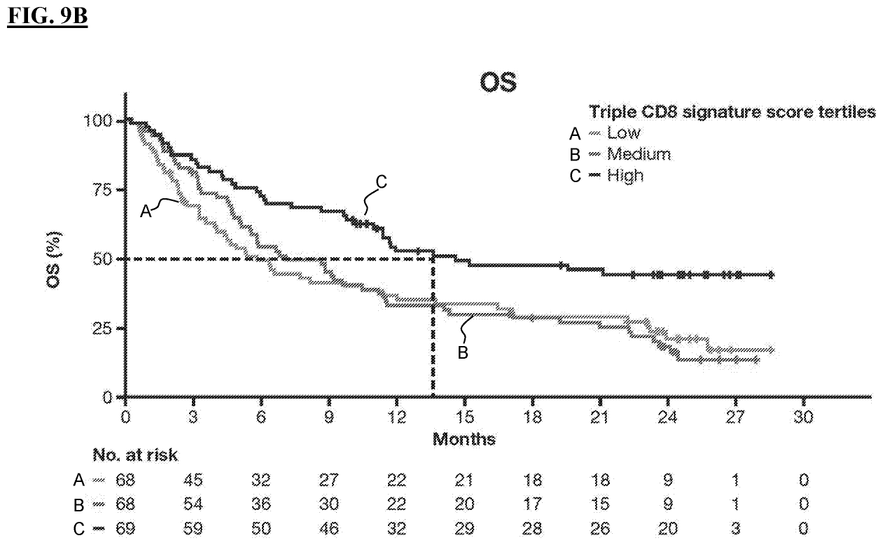

Kaplan-Meier plots illustrate patterns of association between PFS or OS and the triple CD8 signature (FIGS. 9A-9B) that are consistent with those observed by Cox model analyses (FIGS. 8A-8B). PFS and OS with nivolumab were longer for patients with scores in the upper tertile than those in the lower 2 tertiles.

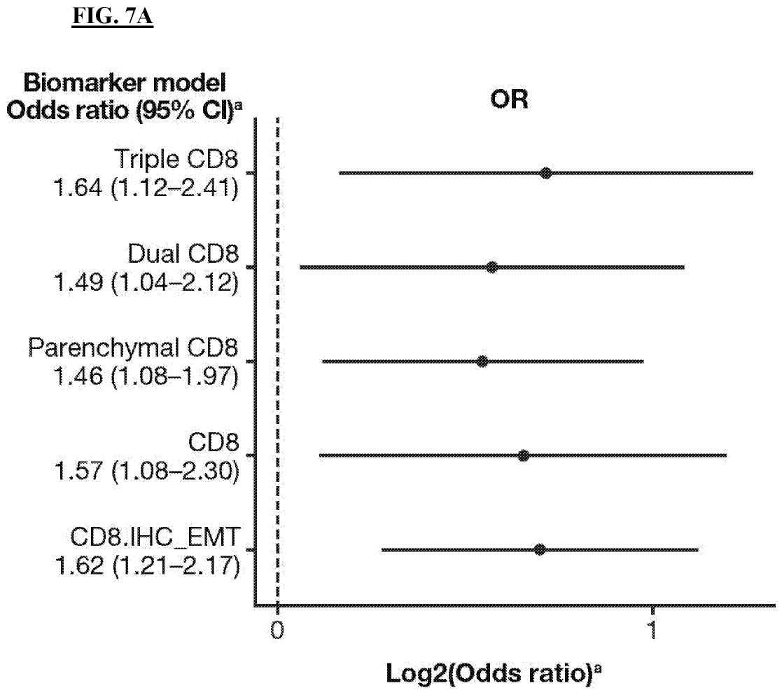

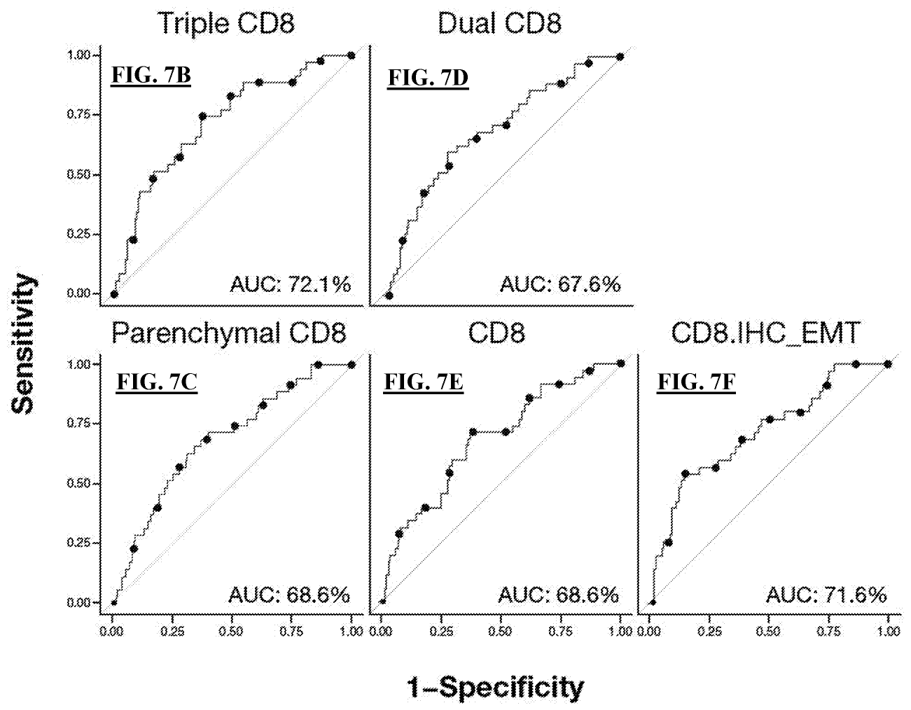

The CD8 signature biomarker models (CD8, parenchymal CD8, dual CD8, and triple CD8) and CD8.IHC_EMT showed similar associations with OR (FIG. 7A). Odds ratios ranged from 1.46 (95% CI, 1.08-1.97) for the parenchymal CD8 signature to 1.64 (95% CI, 1.12-2.41) for triple CD8. ROC curves for OR were similar for each biomarker (FIGS. 7B-7F). AUC values ranged from 67.6% (95% CI, 57.9-77.3) for the dual CD8 signature to 72.1% (95% CI, 62.8-81.4) for triple CD8.

This example demonstrates that CD8 gene signature biomarkers and a CD8 IHC-derived score combined with EMT gene expression (CD8.IHC_EMT) are comparable for the association with response and survival in patients with UC treated with nivolumab. Gene expression signatures associated with CD8+ T-cell localization in the TME may facilitate the selection of patients who are more likely to benefit from I-O therapy. These data illustrate the potential utility of combinatorial biomarkers assessing tumor inflammation for response to I-O therapy in patients with cancer.

What is claimed is:

1. A pharmaceutical composition comprising an anti-PD-1/PD-L1 antagonist for use in a method of identifying a human subject suitable for a combination therapy of the anti-PD-1/PD-L1 antagonist in combination with an anti-cancer agent,
wherein the method comprises measuring expression of a panel of genes in a tumor sample obtained from a subject in need of the combination therapy, wherein the gene panel comprises at least three of CSF1R, NECTIN2, STAT1, and IFNγ.

2. The pharmaceutical composition for use of claim 1, wherein the gene panel comprises at least four, at least five, or at least six of CSF1R, NECTIN2, STAT1, and IFNγ.

3. The pharmaceutical composition for use of claim 1, wherein the gene panel comprises CSF1R, NECTIN2, STAT1, and IFNγ.

4. The pharmaceutical composition for use of any one of claims 1 to 3, wherein the subject is identified as being suitable when the tumor sample exhibits:
(i) an increased expression of one or more of CSF1R and NECTIN2 (“upregulated genes”) in the sample compared to the expression of the one or more of CSF1R and NECTIN2 in a reference sample;
(ii) a decreased expression of one or more of STAT1 and IFNγ (“down-regulated genes”) in the sample compared to the expression of one or more of STAT1 and IFNγ in a reference sample or
(iii) both (i) and (ii).

5. The pharmaceutical composition for use of any one of claims 1 to 5, wherein the subject is to be administered an anti-PD-1/PD-L1 antagonist in combination with an anti-cancer agent.

6. A pharmaceutical composition comprising an anti-PD-1/PD-L1 antagonist in combination with an anti-cancer agent for use in a method of treating a human subject afflicted with a tumor, wherein a tumor sample obtained from the subject exhibits:
(i) an increased expression of one or more of CSF1R and NECTIN2 (“upregulated genes”) in a tumor sample obtained from the subject compared to the expression of the one or more of CSF1R and NECTIN2 in a reference sample;
(ii) a decreased expression of one or more of STAT1 and IFNγ (“down-regulated genes”) in a tumor sample obtained from the subject compared to the expression of one or more of STAT1 and IFNγ in a reference sample; or
(iii) both (i) and (ii).

7. The pharmaceutical composition of any one of claims 4 to 6, wherein the reference sample comprises a non-tumor tissue of the subject, a corresponding non-tumor tissue of the subject, or the corresponding tissue of subjects without a tumor.

8. A method of identifying a human subject suitable for a combination therapy of an anti-PD-1/PD-L1 antagonist in combination with an anti-cancer agent, comprising in vitro measuring expression of a panel of genes in a tumor sample obtained from a subject in need of the anti-PD-1/PD-L1 antagonist, wherein the gene panel comprises at least three of CSF1R, NECTIN2, STAT1, and IFNγ.

9. The method of claim 8, wherein the gene panel comprises at least four, at least five, or at least six of CSF1R, NECTIN2, STAT1, and IFNγ.

10. The method of claim 8, wherein the gene panel comprises CSF1R, NECTIN2, STAT1, and IFNγ.

11. The method of any one of claims 8 to 10, wherein the subject is identified as being suitable when the tumor sample exhibits:
(i) an increased expression of one or more of CSF1R and NECTIN2 (“upregulated genes”) in the tumor sample compared to the expression of the one or more of CSF1R and NECTIN2 in a reference sample;
(ii) a decreased expression of one or more of STAT1 and IFNγ (“down-regulated genes”) in the tumor sample compared to the expression of one or more of STAT1 and IFNγ in a reference sample; or
(iii) both (i) and (ii).

12. The method of claim 11, further comprising administering the anti-PD-1/PD-L1 antagonist in combination with an anti-cancer agent.

13. A method of treating a human subject afflicted with a tumor, comprising administering an anti-PD-1/PD-L1 antagonist to the subject, wherein a tumor sample obtained from the subject exhibit:
(i) an increased expression of one or more of CSF1R and NECTIN2 (“upregulated genes”) in a tumor sample obtained from the subject compared to the expression of the one or more of CSF1R and NECTIN2 in a reference sample;
(ii) a decreased expression of one or more of STAT1 and IFNγ (“down-regulated genes”) in a tumor sample obtained from the subject compared to the expression of one or more of STAT1 and IFNγ in a reference sample; or
(iii) both (i) and (ii).

14. The method of any one of claims 11 to 13, wherein the reference sample comprises a non-tumor tissue of the subject, a corresponding non-tumor tissue of the subject, or the corresponding tissue of subjects without a tumor.

15. The pharmaceutical composition for use of claim 6 or 7 or the method of claim 13 or 14, wherein the subject is identified as being suitable for an the anti-PD-1/PD-L1 antagonist prior to the anti-PD-1/PD-L1 antagonist.

16. The pharmaceutical composition for use of any one of claims 1 to 7 and 15 or the method of any one of claims 8 to 15, wherein the tumor sample exhibits increased expression of at least two of the upregulated genes.

17. The pharmaceutical composition for use of any one of claims 1 to 7, 15, and 16 or the method of any one of claims 8 to 16, wherein the tumor sample exhibits decreased expression of at least two of the down-regulated genes.

18. The pharmaceutical composition for use of any one of claims 1 to 7, and 15 to 17 or the method of any one of claims 8 to 17, wherein the tumor sample exhibits increased expression of all of the upregulated genes; and the tumor sample exhibits decreased expression of all of the down-regulated genes.

19. The pharmaceutical composition for use of any one of claims 1 to 7, and 15 to 18 or the method of any one of claims 11 to 18, wherein the expression of one or more of the upregulated genes is increased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than the expression of one or more of CSF1R and NECTIN2 in the reference sample.

20. The pharmaceutical composition for use of any one of claims 1 to 7, and 15 to 18 or the method of any one of claims 11 to 18, wherein the expression of one or more of the upregulated genes is increased at least about 50% higher than the expression of one or more of CSF1R and NECTIN2 in the reference sample.

21. The pharmaceutical composition for use of any one of claims 1 to 7, and 15 to 18 or the method of any one of claims 11 to 18, wherein the expression of one or more of the upregulated genes is increased at least about 75% higher than the expression of one or more of CSF1R and NECTIN2 in the reference sample.

22. The pharmaceutical composition for use of any one of claims 1 to 7, and 15 to 21 or the method of any one of claims 11 to 21, wherein the expression of one or more of the upregulated genes is decreased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% lower than the expression of one or more of STAT1 and IFNγ in the reference sample.

23. The pharmaceutical composition for use of any one of claims 1 to 7, and 15 to 21 or the method of any one of claims 11 to 21, wherein the expression of one or more of the upregulated genes is decreased at least about 50% lower than the expression of one or more of STAT1 and IFNγ in the reference sample.

24. The pharmaceutical composition for use of any one of claims 1 to 7, and 15 to 21 or the method of any one of claims 11 to 21, wherein the expression of one or more of the upregulated genes is decreased at least about 75% lower than the expression of one or more of STAT1 and IFNγ in the reference sample.

25. The pharmaceutical composition for use of any one of claims 1 to 7, and 15 to 24 or the method of any one of claims 11 to 24, wherein the tumor sample is a tumor tissue biopsy.

26. The pharmaceutical composition for use of any one of claims 1 to 7, and 15 to 25 or the method of any one of claims 11 to 25, wherein the tumor sample is a formalin-fixed, paraffin-embedded tumor tissue or a fresh-frozen tumor tissue.

27. The pharmaceutical composition for use of any one of claims 1 to 7, and 15 to 26 or the method of any one of claims 11 to 26, wherein the tumor sample is obtained from a stroma of the tumor.

28. The pharmaceutical composition for use of any one of claims 1 to 7, and 15 to 27 or the method of any one of claims 11 to 27, wherein gene expression is determined by detecting the presence of gene mRNA, the presence of a protein encoded by the gene, or both.

29. The pharmaceutical composition for use or method of claim 28, wherein the presence of gene mRNA is determined using reverse transcriptase PCR.

30. The pharmaceutical composition for use or method of claim 27 or 28, wherein the presence of the protein encoded by the gene is determined using an IHC assay.

31. The pharmaceutical composition for use or method of claim 29, wherein the IHC assay is an automated IHC assay.

32. The pharmaceutical composition for use or method of claim 32 or 33, wherein the tumor sample is obtained from a stroma of the tumor.

33. The pharmaceutical composition for use of any one of claims 1 to 7, and 15 to 32 or the method of any one of claims 11 to 32, wherein the anti-PD-1/PD-L1 antagonist comprises an antibody or antigen-binding fragment thereof that specifically binds a target protein selected from programmed death 1 (PD-1; an “anti-PD-1 antibody”) or programmed death ligand 1 (PD-L1; an “anti-PD-L1 antibody).

34. The pharmaceutical composition for use or method of claim 33, wherein the anti-PD-1/PD-L1 antagonist is an anti-PD-1 antibody.

35. The pharmaceutical composition for use or method of claim 34, wherein the anti-PD-1 antibody comprises nivolumab or pembrolizumab.

36. The pharmaceutical composition for use or method of claim 33, wherein the anti-PD-1/PD-L1 antagonist is an anti-PD-L1 antibody.

37. The pharmaceutical composition for use or method of claim 34, wherein the anti-PD-1 antibody comprises avelumab, atezolizumab, or durvalumab.

38. The pharmaceutical composition for use of any one of claims 1 to 7, and 15 to 37 or the method of any one of claims 11 to 37, wherein the anti-cancer agent comprises an antibody that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CSF1R, CEACAM-1, CD52, HER2, and any combination thereof.

39. The pharmaceutical composition for use or method of claim 38, wherein the anti-cancer agent comprises an anti-CSF1R antibody.

40. The pharmaceutical composition for use of any one of claims 1 to 7, and 15 to 39 or the method of any one of claims 11 to 39, wherein the tumor is derived from a cancer selected from the group consisting of hepatocellular cancer, gastroesophageal cancer, melanoma, bladder cancer, lung cancer, kidney cancer, head and neck cancer, colon cancer, pancreatic cancer, prostate cancer, ovarian cancer, urothelial cancer, colorectal cancer, and any combination thereof.

41. The pharmaceutical composition for use of any one of claims 1 to 7, and 15 to 40 or the method of any one of claims 11 to 40, wherein the tumor is relapsed.

42. The pharmaceutical composition for use of any one of claims 1 to 7, and 15 to 40 or the method of any one of claims 11 to 40, wherein the tumor is refractory.

43. The pharmaceutical composition for use of any one of claims 1 to 7, and 15 to 40 or the method of any one of claims 11 to 40, wherein the tumor is locally advanced.

44. The pharmaceutical composition for use of any one of claims 1 to 7, and 15 to 40 or the method of any one of claims 11 to 40, wherein the tumor is metastatic.

45. The pharmaceutical composition for use of any one of claims 5 to 7, and 15 to 44 or the method of any one of claims 12 to 44, wherein the administering treats the tumor.

46. The pharmaceutical composition for use of any one of claims 5 to 7, and 15 to 44 or the method of any one of claims 12 to 44, wherein the administering reduces the size of the tumor.

47. The pharmaceutical composition or method of claim 46, wherein the size of the tumor is reduced by at least about 10%, about 20%, about 30%, about 40%, or about 50% compared to the tumor size prior to the administration.

48. The pharmaceutical composition for use of any one of claims 5 to 7, and 15 to 47 or the method of any one of claims 12 to 47, wherein the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

49. The pharmaceutical composition for use of any one of claims 5 to 7, and 15 to 47 or the method of any one of claims 12 to 47, wherein the subject exhibits stable disease after the administration.

50. The pharmaceutical composition for use of any one of claims 5 to 7, and 15 to 47 or the method of any one of claims 12 to 47, wherein the subject exhibits a partial response after the administration.

51. The pharmaceutical composition for use of any one of claims 5 to 7, and 15 to 47 or the method of any one of claims 12 to 47, wherein the subject exhibits a complete response after the administration.

52. A kit for treating a subject afflicted with a tumor, the kit comprising:
(a) an anti-PD-1/PD-L1 antagonist; and
(b) instructions for using the anti-PD-1/PD-L1 antagonist in the pharmaceutical composition in combination with an anti-cancer agent for use of any one of claims 1 to 7, and 15 to 51 or the method of any one of claims 11 to 51.

53. The kit of claim 52, wherein the anti-PD-1/PD-L1 antagonist comprises an anti-PD-1 antibody.

54. The kit of claim 52, wherein the anti-PD-1/PD-L1 antagonist comprises an anti-PD-L1 antibody.

55. The kit of claim 52, wherein the anti-cancer agent comprises an antibody that specifically binds a protein selected from Inducible T cell Co-Stimulator (ICOS), CD137 (4-1BB), CD134 (OX40), NKG2A, CD27, CD96, Glucocorticoid-Induced TNFR-Related protein (GITR), and Herpes Virus Entry Mediator (HVEM), Programmed Death-1 (PD-1), Programmed Death Ligand-1 (PD-L1), CTLA-4, B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), KIR, TGFβ, IL-10, IL-8, B7-H4, Fas ligand, CXCR4, mesothelin, CSF1R, CEACAM-1, CD52, HER2, and any combination thereof.

56. A gene panel comprising at least three of CSF1R, NECTIN2, STAT1, and IFNγ, for use in identifying a subject suitable for a combination therapy comprising an anti-PD-1/PD-L1 antagonist and an anti-cancer agent.

57. The gene panel for use of claim 56, which comprises at least four, at least five, or at least six of CSF1R, NECTIN2, STAT1, and IFNγ.

58. The gene panel for use of claim 57, which comprises CSF1R, NECTIN2, STAT1, and IFNγ.

59. The gene panel for use of claim 56, which consisting of CSF1R, NECTIN2, STAT1, and IFNγ and one additional gene, two additional genes, three additional genes, four additional genes, five additional genes, six additional genes, seven additional genes, eight additional genes, nine additional genes, or ten additional genes.

60. A method for preparing a nucleic acid fraction from a tumor of a subject in need of an I/O therapy, comprising:
(a) extracting a tumor biopsy from the subject;
(b) producing a fraction of nucleic acids extracted in (a) by the isolating nucleic acids; and
(c) analyzing the expression level of one or more genes in a gene panel selected from STAT1, IFNγ, NECTIN2, and CSF1R.

61. The method of claim 60, wherein the nucleic acids are mRNA.

62. The method of claim 60 or 61, wherein one or both of CSF1R and NECTIN2 genes are upregulated.

63. The method of any one of claims 60 to 62, wherein one or both of STAT1 and IFNγ are downregulated.

64. The method of any one of claims 60 to 63, wherein CSF1R and NECTIN2 are upregulated, and wherein STAT1 and IFNγ are downregulated.

65. The method of any one of claims 60 to 64, wherein the expression level of the one or more genes in the gene panel is analyzed by measuring an mRNA level of the one or more genes in the gene panel in the tumor sample.

66. The method of any one of claims 60 to 65, wherein the expression level is measured using a nuclease protection assay.

67. The method of any one of claims 60 to 65, wherein the expression level is measured using next-generation sequencing.

68. The method of any one of claims 60 to 65, wherein the expression level is measured using reverse transcriptase polymerase chain reaction (RT-PCR).

69. The method any one of claims 60 to 68, wherein the expression of one or both of STAT1 and IFNγ is decreased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% lower than the expression of one or both of STAT1 and IFNγ in the reference sample.

70. The method any one of claims 60 to 69, wherein the expression of one or both of STAT1 and IFNγ is decreased at least about 50% lower than the expression of one or both of STAT1 and IFNγ in the reference sample.

71. The method any one of claims 60 to 70, wherein the expression of one or both of STAT1 and IFNγ is decreased at least about 75% lower than the expression of one or both of STAT1 and IFNγ in the reference sample.

72. The method any one of claims 60 to 71, wherein the expression of one or both of NECTIN2 and CSF1R is increased at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than the expression of one or more of NECTIN2 and CSF1R in the reference sample.

73. The method any one of claims 60 to 72 wherein the expression of one or both of NECTIN2 and CSF1R is increased at least about 50% higher than the expression of one or more of NECTIN2 and CSF1R in the reference sample.

74. The method any one of claims 60 to 73, wherein the expression of one or both of NECTIN2 and CSF1R is increased at least about 75% higher than the expression of one or more of NECTIN2 and CSF1R in the reference sample.
